(19) United States
(12) Patent Application Publication
(10) Pub. No.: US 2021/0059251 A1
(43) Pub. Date: Mar. 4, 2021

(54) PLANT GROWTH REGULATOR COMPOUNDS

(71) Applicant: SYNGENTA PARTICIPATIONS AG, Basel (CH)

(72) Inventors: Alexandre Franco Jean Camille LUMBROSO, Stein (CH); Alain DE MESMAEKER, Stein (CH); Claudio SCREPANTI, Stein (CH); Denise Mathilde LACHIA, Stein (CH); Pierre QUINODOZ, Stein (CH)

(73) Assignee: SYNGENTA PARTICIPATIONS AG, Basel (CH)

(21) Appl. No.: 16/980,896

(22) PCT Filed: Mar. 7, 2019
(86) PCT No.: PCT/EP2019/055753

§ 371 Date: Sep. 15, 2020

(30) Foreign Application Priority Data
Mar. 16, 2018 (EP) 1804252.3

(51) Int. Cl. A01N 43/38; C07D 405/12; C07D 417/14; A01N 43/78; C07D 491/048; A01N 43/90

(52) U.S. Cl. CPC A01N 43/38; C07D 405/12; A01N 43/90; A01N 43/78; C07D 491/048; C07D 417/14

(57) ABSTRACT

The present invention relates to relates to novel strigolactam derivatives, to processes for preparing these derivatives including intermediate compounds, to seeds comprising these derivatives, 5 to plant growth regulator or seed germination promoting compositions comprising these derivatives and to methods of using these derivatives in controlling the growth of plants and/or promoting the germination of seeds.

The present invention relates to novel strigolactam derivatives, to processes for preparing these derivatives including intermediate compounds, to seeds comprising these derivatives, to plant growth regulator or seed germination promoting compositions comprising these derivatives and to methods of using these derivatives in controlling the growth of plants and/or promoting the germination of seeds.

Strigolactone derivatives are phytohormones which may have plant growth regulation and seed germination properties. They have previously been described in the literature. Certain known strigolactam derivatives (e.g. see WO2012/080115 and WO2016/193290) may have properties analogous to strigolactones, e.g. plant growth regulation and/or seed germination promotion. For such compounds to be used, in particular, for foliar applications or in seed treatment (e.g. as seed coating components), their binding affinities with the strigolactone receptor D14 are important.

The present invention relates to novel strigolactam derivatives that have improved properties. Benefits of the compounds of the present invention include improved tolerance to abiotic stress, improved seed germination, better regulation of crop growth, improved crop yield, and/or improved physical properties such as chemical, hydrolytic, physical and/or soil stability.

According to the present invention, there is provided a compound of formula (I)

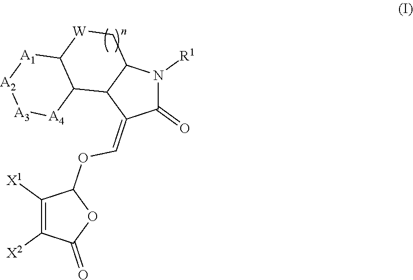

wherein

n is 0, 1 or 2;

W is CH₂ or O;

R¹ is selected from the group consisting of formyl, C₁-C₄ alkylcarbonyl optionally substituted by R², substituted or unsubstituted C₃-C₈ cycloalkylcarbonyl optionally substituted by R², C₁-C₄ alkoxycarbonyl optionally substituted by R², C₁-C₄ haloalkylcarbonyl optionally substituted by R², aryl optionally substituted by R², heteroaryl optionally substituted by R², benzyl optionally substituted by R², and acetonitrile;

A₁ to A₄ are each independently selected from the group consisting of a bond, CR², CR²═CR², C(R²)₂, C(R²)₂—C(R²)₂, N, NR^{B}, S and O; wherein A₁ to A₄ together with the atoms to which they are joined to form a 4 to 7 membered cycloalkyl, heterocycloalkyl, aryl or heteroaryl; and

wherein each R² is independently selected from the group consisting of hydrogen, halogen, C₁-C₄alkyl optionally substituted by R⁸, C₂-C₆ alkenyl optionally substituted by R⁸, C₂-C₆ alkynyl optionally substituted by R⁸, C₁-C₄ alkoxy optionally substituted by R⁸, C₁-C₄ alkoxyalkyl optionally substituted by R⁸, C₁-C₄ hydroxyalkyl optionally substituted by R⁸, and C₁-C₄ haloalkyl optionally substituted by R⁸;

or wherein two R² groups are joined to form a 5-6 membered ring;

wherein each R⁸ is independently selected from the group consisting of hydrogen, halogen, C₁-C₄alkyl, C₂-C₄ alkenyl, C₂-C₄ alkynyl, C₁-C₄ alkoxy, C₃-C₆ cycloalkyl and C₁-C₄ haloalkyl; or two R⁸ groups are joined via —OCH₂O— to form a 5-membered dioxolane ring; and

X¹ and X² are independently selected from the group consisting of hydrogen, C₁-C₄ alkyl, halogen, C₁-C₄ alkoxy, and cyano;

or salts thereof.

The compounds of formula (I) have been shown to possess better affinity with maize strigolactone receptor (D14) as well as improved ability to induce leaf senescence compared to known strigolactam derivatives.

The compounds of the present invention may exist as different geometric isomers (Z or E isomer), optical isomers (diastereoisomers and enantiomers) or tautomeric forms. This invention covers all such isomers and tautomers and mixtures thereof in all proportions as well as isotopic forms such as deuterated compounds. The invention also covers all salts, N-oxides, and metalloidic complexes of the compounds of the present invention.

Each alkyl moiety either alone or as part of a larger group (such as alkoxy, alkoxycarbonyl, alkylcarbonyl, alkylaminocarbonyl, dialkylaminocarbonyl, halogenoalkyl) is a straight or branched chain and is, for example, methyl, ethyl, n-propyl, n-butyl, iso-propyl, n-butyl, sec-butyl, iso-butyl, tert-butyl.

Unless otherwise indicated, cycloalkyl may be mono- or bi-cyclic, may be optionally substituted by one or more C₁-C₆ alkyl groups, and contain 3 to 8 carbon atoms. Examples of cycloalkyl include cyclopropyl, 1-methylcyclopropyl, 2-methylcyclopropyl, cyclobutyl, cyclopentyl and cyclohexyl.

The term “alkenyl”, as used herein, is an alkyl moiety having at least one carbon-carbon double bond, for example C₂-C₆ alkenyl. Specific examples include vinyl and allyl. The alkenyl moiety may be part of a larger group (such as alkenoxy, alkenoxycarbonyl, alkenylcarbonyl, alkyenlaminocarbonyl, dialkenylaminocarbonyl).

The term “alkynyl”, as used herein, is an alkyl moiety having at least one carbon-carbon triple bond, for example C₂-C₆ alkynyl. Specific examples include ethynyl and propargyl. The alkynyl moiety may be part of a larger group (such as alkynoxy, alkynoxycarbonyl, alkynylcarbonyl, alkynylaminocarbonyl, dialkynylaminocarbonyl).

Unless otherwise indicated, alkenyl and alkynyl, on their own or as part of another substituent, may be straight or branched chain and may contain 2 to 6 carbon atoms, and where appropriate, may be in either the (E) or (Z) configuration. Examples include vinyl, allyl, ethynyl and propargyl.

Halogen is fluorine (F), chlorine (Cl), bromine (Br) or iodine (I).

The term “haloalkyl” (either alone or as part of a larger group, such as haloalkoxy or haloalkylthio), as used herein, are alkyl groups which are substituted with one or more of the same or different halogen atoms and are, for example, —CF₃, —CF₂Cl, —CH₂CF₃ or —CH₂CHF₂.

The term “hydroxyalkyl” as used herein, are alkyl groups which are substituted with one or more hydroxyl group and are, for example, —CH₂OH, —CH₂CH₂OH or —CH(OH)CH₃.

The term “alkoxyalkyl”, as used herein are alkoxy groups bonded to an alkyl (R—O—R′), for example —(CH₂)rO(CH₂)sCH₃, wherein r is 1 to 6 and s is 1 to 5.

The term “aryl”, as used herein, refers to a ring system which may be mono, bi or tricyclic. Examples of such rings include phenyl, naphthalenyl, anthracenyl, indenyl or phenanthrenyl.

The term “heteroaryl”, as used herein, refers to an aromatic ring system containing from one to four heteroatoms selected from N, O, and S, wherein the nitrogen and sulfur atoms are optionally oxidized, for example having 5, 6, 9 or 10 members, and consisting either of a single ring or of two or more fused rings. Single rings may contain up to three heteroatoms, and bicyclic systems up to four heteroatoms, which will preferably be chosen from nitrogen, oxygen and sulfur. Examples of such groups include pyridyl, pyridazinyl, pyrimidinyl, pyrazinyl, furanyl, thienyl, oxazolyl, isoxazolyl, oxadiazolyl, thiazolyl, isothiazolyl, thiadiazolyl, pyrrolyl, pyrazolyl, imidazolyl, triazolyl and tetrazolyl.

According to the present invention, there is provided a compound of formula II

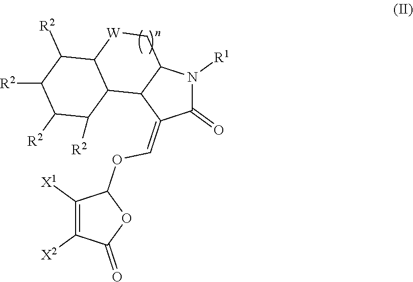

wherein

n is 0, 1 or 2;

W is CH₂ or O;

R¹ is selected from the group consisting of formyl, C₁-C₄ alkylcarbonyl optionally substituted by R², C₃-C₈ cycloalkylcarbonyl optionally substituted by R², C₁-C₄alkoxycarbonyl optionally substituted by R², C₁-C₄ haloalkylcarbonyl optionally substituted by R², aryl optionally substituted by R², heteroaryl optionally substituted by R², benzyl optionally substituted by R², and acetonitrile;

wherein each R² is independently selected from the group consisting of hydrogen, halogen, C₁-C₄alkyl optionally substituted by R⁸, C₂-C₆ alkenyl optionally substituted by R⁸, C₂-C₆ alkynyl optionally substituted by R⁸, C₁-C₄ alkoxy optionally substituted by R⁸, C₁-C₄ alkoxyalkyl optionally substituted by R⁸, C₁-C₄ hydroxyalkyl optionally substituted by R⁸, and C₁-C₄ haloalkyl optionally substituted by R⁸;

or wherein two R² groups are joined to form a 5-6 membered ring;

wherein each R⁸ is independently selected from the group consisting of hydrogen, halogen, C₁-C₄alkyl, C₂-C₄ alkenyl, C₂-C₄ alkynyl, C₁-C₄ alkoxy, C₃-C₆ cycloalkyl and C₁-C₄ haloalkyl; or two R⁸ groups are joined via —OCH₂O— to form a 5-membered dioxolane ring; and

X¹ and X² are independently selected from the group consisting of hydrogen, C₁-C₄ alkyl, halogen, C₁-C₄ alkoxy, and cyano;

or salts thereof.

Further definitions of W, X¹, X², R¹, R², A₁, A₂, A₃ and A₄ are, in any combination, as set out below.

W in compounds of the present invention is a carbon, or an oxygen. In one embodiment, W is carbon. In another embodiment W is oxygen.

X¹ and X² in compounds of the present invention are independently selected from the group consisting of hydrogen, C₁-C₃alkyl and C₁-C₃alkoxy. In one embodiment, X¹ and X² are independently selected from the group consisting of hydrogen, methyl, ethyl and methoxy. In a further embodiment, X¹ and X² are independently selected from the group consisting of hydrogen and methyl.

In one embodiment X¹ is hydrogen or methyl and X² is methyl. In a further embodiment, X¹ is methyl and X² is methyl.

A₁ to A₄ in compounds of the invention are each independently selected from the group consisting of a bond, CR², CR²═CR², C(R²)₂, C(R²)₂—C(R²)₂, N, NR⁸, S and O, wherein A₁ to A₄ together with the atoms to which they are joined form a 4 to 7 membered cycloalkyl, heterocycloalkyl, aryl or heteroaryl; and each R⁸ is independently selected from the group consisting of hydrogen, halogen, C₁-C₄alkyl, C₂-C₄ alkenyl, C₂-C₄ alkynyl, C₁-C₄ alkoxy and C₁-C₄ haloalkyl; or two R⁸ groups are joined via —OCH₂O— to form a 5-membered dioxolane ring.

In one embodiment A₁ to A₄ are each independently selected from the group consisting of a bond, CR², N, S and O, wherein A₁ to A₄ together with the atoms to which they are joined form a 5 to 7 membered cycloalkyl such as cyclopentyl, cyclohexyl, cycloheptyl; or an aryl ring such as phenyl.

In one embodiment, the ring formed by A₁ to A₄ is a phenyl, pyrimidine or pyrazine ring, each optionally substituted with 1-4 R².

In one embodiment A₁ to A₄ together with the atoms to which they are joined form a phenyl ring substituted with 1-4 R².

In one embodiment R² is independently selected from the group consisting of hydrogen, halogen, C₁-C₃ alkyl, C₁-C₃ alkoxy and C₁-C₃ haloalkyl; or two R² groups are joined via —OCH₂O— to form a 5-membered dioxolane ring.

In one embodiment R² is selected from the group consisting of hydrogen, fluoro, chloro, methyl, ethyl, methoxy, ethoxy, fluoromethyl and trifluoromethyl.

In one embodiment at least one R² is selected from the group consisting of, fluoro, chloro, methyl, ethyl, methoxy, ethoxy, fluoromethyl and trifluoromethyl.

In one embodiment one R² is selected from the group consisting of, fluoro, chloro, methyl, ethyl, methoxy, ethoxy, fluoromethyl and trifluoromethyl.

In one embodiment, each R² is hydrogen.

In one embodiment, R¹ is selected from the group consisting of formyl, C₁-C₄ alkylcarbonyl optionally substituted by one or more R², C₃-C₆ cycloalkylcarbonyl optionally substituted by one or more R², C₁-C₄ alkoxycarbonyl optionally substituted by one or more R², C₁-C₄ haloalkylcarbonyl optionally substituted by one or more R², aryl optionally substituted by one or more R², heteroaryl optionally substituted by one or more R², benzyl optionally substituted by one or more R², and acetonitrile.

In one embodiment, R¹ is selected from the group consisting of formyl, C₁-C₄ alkylcarbonyl optionally substituted by R², C₃-C₆ cycloalkylcarbonyl optionally substituted by R², C₁-C₄ alkoxycarbonyl optionally substituted by R², C₁-C₄ haloalkylcarbonyl optionally substituted by R², aryl optionally substituted by R², heteroaryl optionally substituted by R², benzyl optionally substituted by R², and acetonitrile.

In one embodiment, R¹ is selected from the group consisting of formyl, C₁-C₄ alkylcarbonyl, C₃-C₆ cycloalkylcarbonyl, C₁-C₄ alkoxycarbonyl, C₁-C₄ haloalkylcarbonyl, aryl, heteroaryl, benzyl, and acetonitrile.

In a further embodiment, R¹ is selected from the group consisting of phenyl, C₁-C₄alkylcarbonyl, heteroaryl, and acetonitrile.

In a still further embodiment, R¹ is selected from the group consisting of phenyl, acetyl, thiazolyl, and acetonitrile.

In one embodiment, R⁸ is independently selected from the group consisting of hydrogen, halogen, C₁-C₄ alkyl, C₂-C₄ alkenyl, C₂-C₄ alkynyl, C₁-C₄ alkoxy, C₃-C₆ cycloalkyl and C₁-C₄ haloalkyl.

In a further embodiment, R⁸ is independently selected from the group consisting of hydrogen, halogen, and C₁-C₄ alkyl.

In one embodiment, R⁸ is H.

In one embodiment of the present invention there is provided a compound of formula (I) or (II) wherein X¹ is hydrogen or methyl; X² is methyl; R¹ is selected from the group consisting of phenyl, C₁-C₄alkylcarbonyl, heteroaryl, and acetonitrile; A₁ to A₄ together with the atoms to which they are joined form a phenyl optionally substituted with 1-4 R²; and each R² is independently selected from the group consisting of hydrogen, halogen, C₁-C₃ alkyl, C₁-C₃ alkoxy and C₁-C₃ haloalkyl; or two R² groups are joined via —OCH₂O— to form a 5-membered dioxolane ring.

In a further embodiment, there is provided a compound of formula (I) or (II) wherein X¹ is hydrogen or methyl; X² is methyl; R¹ is selected from the group consisting of phenyl, acetyl, thiazolyl, and acetonitrile; A₁ to A₄ together with the atoms to which they are joined form a phenyl optionally substituted with 1-4 R²; and each R² is independently selected from the group consisting of hydrogen, halogen, C₁-C₃ alkyl, C₁-C₃ alkoxy and C₁-C₃ haloalkyl; or two R² groups are joined via —OCH₂O— to form a 5-membered dioxolane ring.

Compounds of formula (Ia) to (Id) represent specific embodiments of the present invention:

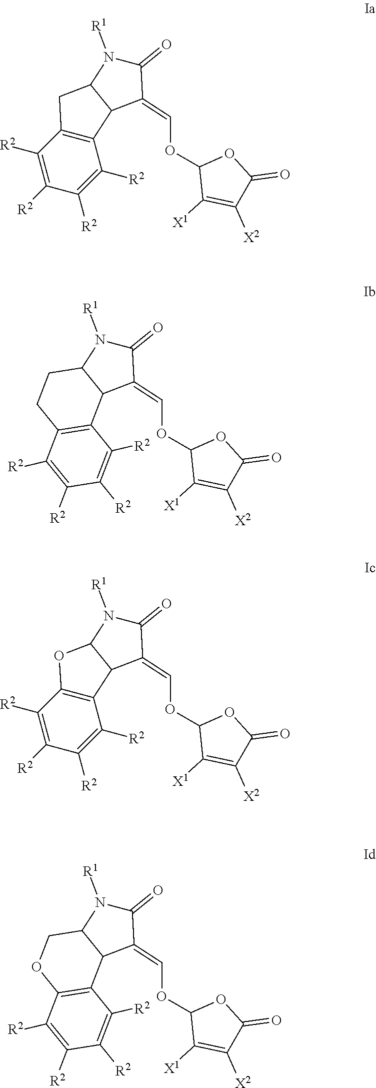

The definitions of X¹, X², R¹, and R² are, in any combination, as described above.

According to the present invention there is provided a compound of formula (Ia), (Ib), (Ic) or (Id) wherein X¹ is hydrogen or methyl and X² is methyl.

According to the present invention there is provided a compound of formula (Ia), (Ib), (Ic) or (Id) wherein R¹ is selected from the group consisting of thiazolyl, phenyl, acetonitrile, CH₃(CO)—, C₂H₅(CO)—, C₃H₇(CO)—, C₃H₅(CO)—, CF₃(CO)—, and CF₃CH₂(CO).

According to the present invention there is provided a compound of formula (Ia), (Ib), (Ic) or (Id) wherein R² are all CH.

TABLE 1
Examples of specific compounds of the present
invention. R^{2a}, R^{2b}, R^{2c} and R^{2d}represent
the four available substitution positions on the phenyl ring.

| Com- | Ring | | | | | | | |
|---|---|---|---|---|---|---|---|---|
| | sys- | | | | | | | |
| pound | tem | R¹ | R^{2a} | R^{2b} | R^{2c} | R^{2d} | X¹ | X² |
| I-1 | Ia | CH₃(CO)— | H | H | H | H | H | Me |
| I-2 | Ia | C₂H₅(CO)— | H | H | H | H | H | Me |
| I-3 | Ia | C₃H₇(CO)— | H | H | H | H | H | Me |
| I-4 | Ia | C₃H₅(CO)— | H | H | H | H | H | Me |
| I-5 | Ia | CH₃(CO)— | H | H | H | H | Me | Me |
| I-6 | Ia | C₂H₅(CO)— | H | H | H | H | Me | Me |
| I-7 | Ia | C₃H₇(CO)— | H | H | H | H | Me | Me |
| I-8 | Ia | C₃H₅(CO)— | H | H | H | H | Me | Me |
| I-9 | Ia | Thiazolyl | H | H | H | H | H | Me |
| I-10 | Ia | Phenyl | H | H | H | H | H | Me |
| I-11 | Ia | 3,5-(CF₃)₂Ph | H | H | H | H | H | Me |
| I-12 | Ia | CH₂CN | H | H | H | H | H | Me |
| I-13 | Ia | Thiazolyl | H | H | H | H | Me | Me |
| I-14 | Ia | Phenyl | H | H | H | H | Me | Me |
| I-15 | Ia | 3,5-(CF₃)₂Ph | H | H | H | H | Me | Me |
| I-16 | Ia | CH₂CN | H | H | H | H | Me | Me |
| I-17 | Ia | CF₃(CO)— | H | H | H | H | H | Me |
| I-18 | Ia | CF₃CH₂ (CO)— | H | H | H | H | H | Me |
| I-19 | Ia | CF₃(CO)— | H | H | H | H | Me | Me |
| I-20 | Ia | CF₃CH₂(CO)— | H | H | H | H | Me | Me |
| I-21 | Ic | CH₃(CO)— | H | H | H | H | H | Me |
| I-22 | Ic | C₂H₅(CO)— | H | H | H | H | H | Me |
| I-23 | Ic | C₃H₇(CO)— | H | H | H | H | H | Me |
| I-24 | Ic | C₃H₅(CO)— | H | H | H | H | H | Me |
| I-25 | Ic | CH₃(CO)— | H | H | H | H | Me | Me |
| I-26 | Ic | C₂H₅(CO)— | H | H | H | H | Me | Me |
| I-27 | Ic | C₃H₇(CO)— | H | H | H | H | Me | Me |
| I-28 | Ic | C₃H₅(CO)— | H | H | H | H | Me | Me |
| I-29 | Ic | Thiazolyl | H | H | H | H | H | Me |
| I-30 | Ic | Phenyl | H | H | H | H | H | Me |
| I-31 | Ic | 3,5-(CF₃)₂Ph | H | H | H | H | H | Me |
| I-32 | Ic | CH₂CN | H | H | H | H | H | Me |
| I-33 | Ic | Thiazolyl | H | H | H | H | Me | Me |
| I-34 | Ic | Phenyl | H | H | H | H | Me | Me |
| I-35 | Ic | 3,5-(CF₃)₂Ph | H | H | H | H | Me | Me |
| I-36 | Ic | CH₂CN | H | H | H | H | Me | Me |
| I-37 | Ic | CF₃(CO)— | H | H | H | H | H | Me |
| I-38 | Ic | CF₃CH₂(CO)— | H | H | H | H | H | Me |
| I-39 | Ic | CF₃(CO)— | H | H | H | H | Me | Me |
| I-40 | Ic | CF₃CH₂(CO)— | H | H | H | H | Me | Me |
| I-41 | Ib | CH₃(CO)— | H | H | H | H | H | Me |
| I-42 | Ib | C₂H₅(CO)— | H | H | H | H | H | Me |
| I-43 | Ib | C₃H₇(CO)— | H | H | H | H | H | Me |
| I-44 | Ib | C₃H₅(CO)— | H | H | H | H | H | Me |
| I-45 | Ib | CH₃(CO)— | H | H | H | H | Me | Me |
| I-46 | Ib | C₂H₅(CO)— | H | H | H | H | Me | Me |
| I-47 | Ib | C₃H₇(CO)— | H | H | H | H | Me | Me |
| I-48 | Ib | C₃H₅(CO)— | H | H | H | H | Me | Me |
| I-49 | Ib | Thiazolyl | H | H | H | H | H | Me |
| I-50 | Ib | Phenyl | H | H | H | H | H | Me |
| I-51 | Ib | 3,5-(CF₃)₂Ph | H | H | H | H | H | Me |
| I-52 | Ib | CH₂CN | H | H | H | H | H | Me |
| I-53 | Ib | Thiazolyl | H | H | H | H | Me | Me |
| I-54 | Ib | Phenyl | H | H | H | H | Me | Me |
| I-55 | Ib | 3,5-(CF₃)₂Ph | H | H | H | H | Me | Me |
| I-56 | Ib | CH₂CN | H | H | H | H | Me | Me |
| I-57 | Ib | CF₃(CO)— | H | H | H | H | H | Me |
| I-58 | Ib | CF₃CH₂(CO)— | H | H | H | H | H | Me |
| I-59 | Ib | CF₃(CO)— | H | H | H | H | Me | Me |
| I-60 | Ib | CF₃CH₂(CO)— | H | H | H | H | Me | Me |
| I-61 | Id | CH₃(CO)— | H | H | H | H | H | Me |
| I-62 | Id | C₂H₅(CO)— | H | H | H | H | H | Me |
| I-63 | Id | C₃H₇(CO)— | H | H | H | H | H | Me |
| I-64 | Id | C₃H₅(CO)— | H | H | H | H | H | Me |
| I-65 | Id | CH₃(CO)— | H | H | H | H | Me | Me |
| I-66 | Id | C₂H₅(CO)— | H | H | H | H | Me | Me |
| I-67 | Id | C₃H₇(CO)— | H | H | H | H | Me | Me |
| I-68 | Id | C₃H₅(CO)— | H | H | H | H | Me | Me |
| I-69 | Id | Thiazolyl | H | H | H | H | H | Me |
| I-70 | Id | Phenyl | H | H | H | H | H | Me |
| I-71 | Id | 3,5-(CF₃)₂Ph | H | H | H | H | H | Me |
| I-72 | Id | CH₂CN | H | H | H | H | H | Me |
| I-73 | Id | Thiazolyl | H | H | H | H | Me | Me |
| I-74 | Id | Phenyl | H | H | H | H | Me | Me |
| I-75 | Id | 3,5-(CF₃)₂Ph | H | H | H | H | Me | Me |
| I-76 | Id | CH₂CN | H | H | H | H | Me | Me |
| I-77 | Id | CF₃(CO)— | H | H | H | H | H | Me |
| I-78 | Id | CF₃CH₂(CO)— | H | H | H | H | H | Me |
| I-79 | Id | CF₃(CO)— | H | H | H | H | Me | Me |
| I-80 | Id | CF₃CH₂(CO)— | H | H | H | H | Me | Me |
| I-81 | Ia | tBuO(CO)— | H | H | H | H | H | Me |
| I-82 | Ia | tBuO(CO)— | H | H | H | H | Me | Me |
| I-83 | Ic | tBuO(CO)— | H | H | H | H | H | Me |
| I-84 | Ic | tBuO(CO)— | H | H | H | H | Me | Me |
| I-85 | Ib | tBuO(CO)— | H | H | H | H | H | Me |
| I-86 | Ib | tBuO(CO)— | H | H | H | H | Me | Me |
| I-87 | Id | tBuO(CO)— | H | H | H | H | H | Me |
| I-88 | Id | tBuO(CO)— | H | H | H | H | Me | Me |
| I-89 | Ia | MeO—CH₂—(CO)— | H | H | H | H | H | Me |
| I-90 | Ia | MeO—CH₂—(CO)— | H | H | H | H | Me | Me |
| I-91 | Ia | (CH₃)₂—CH—CH₂—(CO)— | H | H | H | H | H | Me |
| I-92 | Ia | (CH₃)₂—CH—CH₂—(CO)— | H | H | H | H | Me | Me |
| I-93 | Ia | MeO(CO)— | H | H | H | H | H | Me |
| I-94 | Ia | MeO(CO)— | H | H | H | H | Me | Me |

In one embodiment, the compounds of the present invention are applied in combination with an agriculturally acceptable adjuvant. In particular, there is provided a composition comprising a compound of the present invention and an agriculturally acceptable adjuvant. There may also be mentioned an agrochemical composition comprising a compound of the present invention.

The present invention provides a method of improving the tolerance of a plant to abiotic stress, wherein the method comprises applying to the plant, plant part, plant propagation material, or plant growing locus a compound, composition or mixture according to the present invention.

The present invention provides a method for regulating or improving the growth of a plant, wherein the method comprises applying to the plant, plant part, plant propagation material, or plant growing locus a compound, composition or mixture according to the present invention. In one embodiment, plant growth is regulated or improved when the plant is subject to abiotic stress conditions.

The present invention also provides a method for improving the hydrolytic conductivity of a plant, wherein the method comprises applying to the plant, plant part, plant propagation material, or plant growing locus a compound, composition or mixture according to the present invention.

The present invention also provides a method for promoting seed germination of a plant, comprising applying to the seed, or a locus containing seeds, a compound, composition or mixture according to the present invention.

The present invention also provides a method for controlling weeds comprising applying to a locus containing weed seeds a seed germination promoting amount of a compound, composition or mixture according to the present invention, allowing the seeds to germinate, and then applying to the locus a post-emergence herbicide. The present invention also provides a method for safening a plant against phytotoxic effects of chemicals, comprising applying to the plant, plant part, plant propagation material, or plant growing locus a compound, composition or mixture according to the present invention.

In a further aspect of the invention, there is provided the use of a compound of formula (I) or (II) according to the invention as a crop yield enhancer, plant growth regulator or a seed germination promoter.

The present invention also provides a method for accelerating senescence of plant leaves, comprising applying to the plant, plant part, plant propagation material, or plant growing locus a compound, composition or mixture according to the present invention. In one embodiment, the compound, composition or mixture of the present invention is applied in a leaf senescence regulating amount.

Suitably the compound or composition is applied in an amount sufficient to elicit the desired response.

In a further aspect of the invention, there is provided a method of treating a plant propagation material comprising applying to the plant propagation material a composition according to the invention in an amount effective to promote germination, to enhance the yield and/or regulate plant growth.

In a further aspect of the invention, there is provided a plant propagation material treated with a compound of formula (I) or (II) according to the invention, or a composition according to the invention.

The present invention may also provide method to improve nutrient (such as nitrogen or sugar) recycling and remobilization in plants via leaf senescence.

According to the present invention, “regulating or improving the growth of a crop” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use less seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, less dead basal leaves, delay of senescence, improved vitality of the plant, increased levels of amino acids in storage tissues and/or less inputs needed (e.g. less fertiliser, water and/or labour needed). A plant with improved vigour may have an increase in any of the aforementioned traits or any combination or two or more of the aforementioned traits.

An ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants)), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to biotic and/or abiotic stress factors, and in particular abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve tolerance to drought stress.

An ‘improved input use efficiency’ means that the plants are able to grow more effectively using given levels of inputs compared to the grown of control plants which are grown under the same conditions in the absence of the method of the invention. In particular, the inputs include, but are not limited to fertiliser (such as nitrogen, phosphorous, potassium, micronutrients), light and water. A plant with improved input use efficiency may have an improved use of any of the aforementioned inputs or any combination of two or more of the aforementioned inputs.

Other effects of regulating or improving the growth of a crop include a decrease in plant height, or reduction in tillering, which are beneficial features in crops or conditions where it is desirable to have less biomass and fewer tillers.

Any or all of the above crop enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g. perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The compounds of the present invention can be used alone, but are generally formulated into compositions using formulation adjuvants, such as carriers, solvents and surface-active agents (SFAs). Thus, the present invention further provides a composition comprising a compound of the present invention and an agriculturally acceptable formulation adjuvant. There is also provided a composition consisting essentially of a compound of the present invention and an agriculturally acceptable formulation adjuvant. There is also provided a composition consisting of a compound of the present invention and an agriculturally acceptable formulation adjuvant.

The present invention further provides a crop yield enhancing composition comprising a compound of the present invention and an agriculturally acceptable formulation adjuvant. There is also provided a crop yield enhancing composition consisting essentially of a compound of the present invention and an agriculturally acceptable formulation adjuvant. There is also provided a crop yield enhancing composition consisting of a compound of the present invention and an agriculturally acceptable formulation adjuvant.

In one aspect of the invention, there is provided a crop yield enhancing, abiotic stress management, plant growth regulator or seed germination promoting composition, comprising a compound of the present invention, and optionally, an agriculturally acceptable formulation adjuvant.

The present invention further provides a plant growth regulator composition comprising a compound of the present invention and an agriculturally acceptable formulation adjuvant. There is also provided a plant growth regulator composition consisting essentially of a compound of the present invention and an agriculturally acceptable formulation adjuvant. There is also provided a plant growth regulator composition consisting of a compound of the present invention and an agriculturally acceptable formulation adjuvant.

The present invention further provides a plant abiotic stress management composition comprising a compound of the present invention and an agriculturally acceptable formulation adjuvant. There is also provided a plant abiotic stress management composition consisting essentially of a compound of the present invention and an agriculturally acceptable formulation adjuvant. There is also provided a plant abiotic stress management composition consisting of a compound of the present invention and an agriculturally acceptable formulation adjuvant.

The present invention further provides a seed germination promoting composition comprising a compound of the present invention and an agriculturally acceptable formulation adjuvant. There is also provided a seed germination promoting composition consisting essentially of a compound of the present invention and an agriculturally acceptable formulation adjuvant. There is also provided a seed germination promoting composition consisting of a compound of the present invention and an agriculturally acceptable formulation adjuvant.

The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The compositions generally comprise from 0.1 to 99% by weight, especially from 0.1 to 95% by weight, compounds of the present invention and from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultralow volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of the present invention.

Dustable powders (DP) may be prepared by mixing a compound of the present invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of the present invention with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the present invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the present invention and one or more powdered solid diluents or carriers, or from pre-formed blank granules by absorbing a compound of the present invention (or a solution thereof, in a suitable agent) in a porous granular material (such as pumice, attapulgite clays, fuller's earth, kieselguhr, diatomaceous earths or ground corn cobs) or by adsorbing a compound of the present invention (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the present invention in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of the present invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of the present invention either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the present invention is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the present invention. SCs may be prepared by ball or bead milling the solid compound of the present invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the present invention may be dry milled and added to water, containing agents hereinbefore described, to produce the desired end product.

Aerosol formulations comprise a compound of the present invention and a suitable propellant (for example n-butane). A compound of the present invention may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of the present invention and, optionally, a carrier or diluent therefor. The polymeric shell may be produced by either an interfacial polycondensation reaction or by a coacervation procedure. The compositions may provide for controlled release of the compound of the present invention and they may be used for seed treatment. A compound of the present invention may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of the present invention. Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of the present invention).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The compound or composition of the present invention may be applied to a plant, part of the plant, plant organ, plant propagation material or a plant growing locus.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The application is generally made by spraying the composition, typically by tractor mounted sprayer for large areas, but other methods such as dusting (for powders), drip or drench can also be used. Alternatively the composition may be applied in furrow or directly to a seed before or at the time of planting.

The compound or composition of the present invention may be applied pre-emergence or post-emergence. Suitably, where the composition is used to regulate the growth of crop plants or enhance the tolerance to abiotic stress, it may be applied post-emergence of the crop. Where the composition is used to promote the germination of seeds, it is applied pre-emergence.

The present invention envisages application of the compounds or compositions of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field; removed from the plant; and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively or in addition active ingredients may be applied on a suitable substrate sown together with the plant propagation material.

The rates of application of compounds of the present invention may vary within wide limits and depend on the nature of the soil, the method of application (pre- or post-emergence; seed dressing; application to the seed furrow; no tillage application etc.), the crop plant, the prevailing climatic conditions, and other factors governed by the method of application, the time of application and the target crop. For foliar or drench application, the compounds of the present invention according to the invention are generally applied at a rate of from 1 to 2000 g/ha, especially from 5 to 1000 g/ha. For seed treatment the rate of application is generally between 0.0005 and 150 g per 100 kg of seed.

The compounds and compositions of the present invention may be applied to dicotyledonous or monocotyledonous crops. Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield@ summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 6-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Compounds of the present invention may also be used to promote the germination of seeds of non-crop plants, for example as part of an integrated weed control program.

Normally, in the management of a crop a grower would use one or more other agronomic chemicals or biologicals in addition to the compound or composition of the present invention. There is also provided a mixture comprising a compound or composition of the present invention, and a further active ingredient.

Examples of agronomic chemicals or biologicals include pesticides, such as acaricides, bactericides, fungicides, herbicides, insecticides, nematicides, plant growth regulators, crop enhancing agents, safeners as well as plant nutrients and plant fertilizers. Examples of suitable mixing partners may be found in the Pesticide Manual, 15th edition (published by the British Crop Protection Council).

Such mixtures may be applied to a plant, plant propagation material or plant growing locus either simultaneously (for example as a pre-formulated mixture or a tank mix), or sequentially in a suitable timescale. Co-application of pesticides with the present invention has the added benefit of minimising farmer time spent applying products to crops. The combination may also encompass specific plant traits incorporated into the plant using any means, for example conventional breeding or genetic modification.

The present invention also provides the use of a compound of formula (I), (Ia), (Ib), (Ic), (Id) or (II) or a composition comprising a compound according to formula (I), (Ia), (Ib), (Ic), (Id) or (II) and an agriculturally acceptable formulation adjuvant, for improving the tolerance of a plant to abiotic stress, regulating or improving the growth of a plant, promoting seed germination and/or safening a plant against phytotoxic effects of chemicals.

There is also provided the use of a compound, composition or mixture of the present invention, for improving the tolerance of a plant to abiotic stress, regulating or improving the growth of a plant, promoting seed germination and/or safening a plant against phytotoxic effects of chemicals.

The compounds of the invention may be made by the following methods.

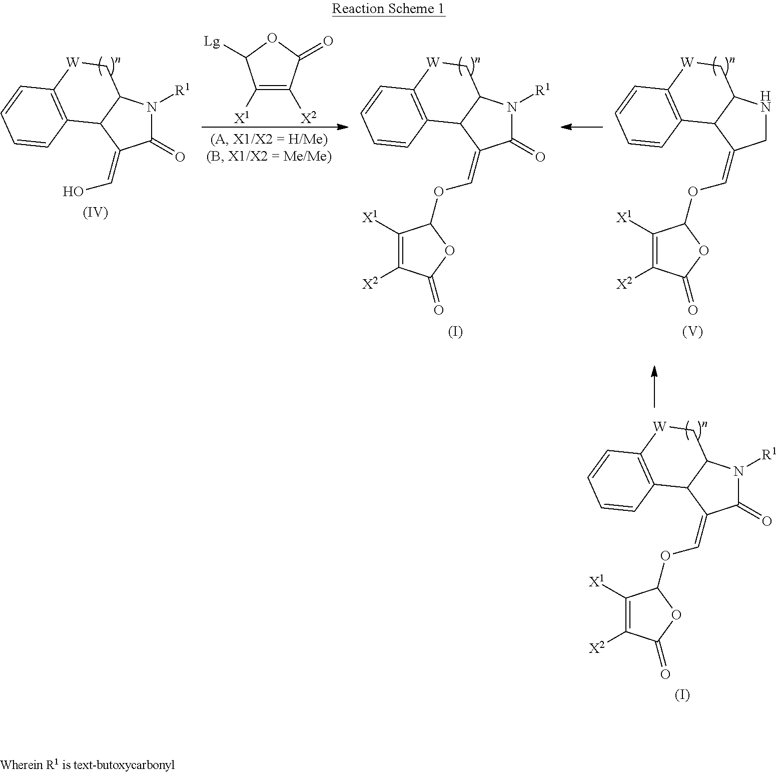

Compounds of formula (I) may be prepared from compounds of formula (IV) by reaction with a compound of formula (IV) and compound (A or B) in the presence of a base such potassium tert-butylate or sodium tert-butylate, in the presence or not of a crown ether to activate the base. The reaction can also be carried out in the presence of a catalytic or stoichiometric amount of iodine salt, such as potassium iodide or tetrabutyl ammonium iodide. Compounds of formula (I) can be prepared by a method similar to what is described in WO2012/080115.

Alternatively, compound (I), wherein R¹ is alkylcarbonyl, can be prepared from compound of formula (V) by reaction with an acychloride or an anhydride (R¹=alkylcarbonyl) in presence of a base such as pyridine, trimethylamine or diisopropyl, ethyl amine and in some cases dimethyl aminopyridine (DMAP). Compounds of formula (V) may be prepared from a compound of formula (I) wherein R¹ is an alkoxycarbonyl group such as tert-butoxycarbonyl, by reaction with an organic or inorganic acid such as trifluoroacetic acid or HCl, or in the presence of a Lewis acid such as a magnesium salt.

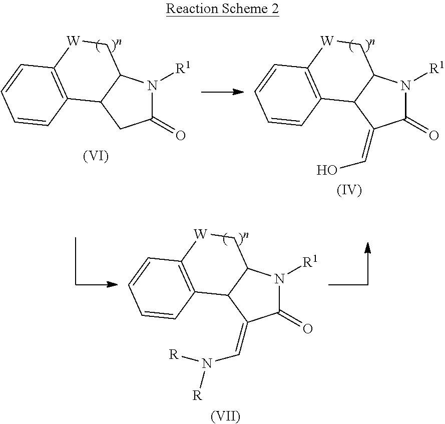

Compounds of formula (IV) may be prepared from a compound of formula (VI) via reaction with a formic ester derivative such as the methyl formate in presence of base such as lithium diisopropylamide, potassium tert-butylate or sodium tert-butylate. Alternatively, compounds of formula (IV) may be prepared from a compound of formula (VII) via hydrolysis with an acid such as hydrogen chloride. Compounds of formula (VII) may be prepared from a compound of formula (VI) via reaction with Bredereck's reagent (tert-butoxybis(dimethylamino)methane) wherein R is a methyl or analogue. Compounds of formula (IV) can be prepared by a method similar to what is described in WO2012/080115.

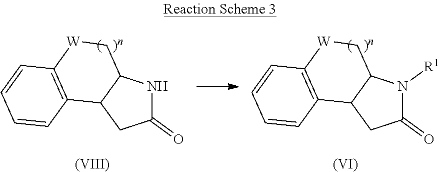

Compounds of formula (VI) can be prepared from a compound of formula (VIII) by treatment with an halogeno-aryl such as phenyl iodide, a halogeno-heteroaryl such as 2-bromothiazol, an anhydride such as acetic anhydride, an acyl chloride and an halogeno-acetonitrile such as 2-bromoacetonitrile in presence of a suitable catalyst and/or base as used in methods described in WO2012/080115

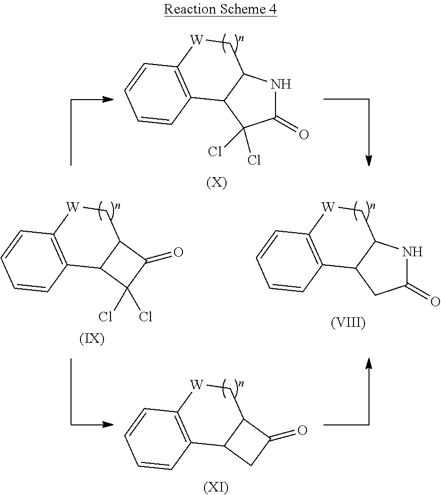

Compounds of formula (VIII) may be prepared from a compound of formula (X) via reduction reaction using an organic or inorganic acid such as ammonium chloride and a metal source such as Zinc. Compound of formula (X) may be prepared from compound of formula (IX) via Baeyer-Villiger reaction (X═O) using a peroxide such as Magnesium monoperoxyphthalate (MMPP) or via Beckmann reaction (X═NR¹) using mesityl sulfonyl hydroxylamine (MSH) or hydroxylamine. Alternatively, compound of formula (VIII) may be prepared form compound of formula (XI) via Bayer-Villiger reaction (X═O) using a peroxide such as Magnesium monoperoxyphthalate (MMPP) or via Beckmann reaction (X═NR¹) using mesityl sulfonyl hydroxylamine (MSH) or hydroxylamine. Compound of formula (XI) may be prepared from compound of formula (IX) via reduction reaction using an acid such as ammonium chloride and a metal such as Zinc

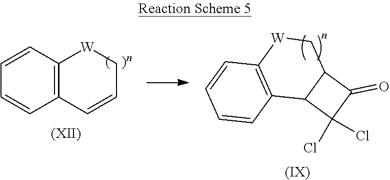

Compounds of formula (IX) may be prepared form commercially available compound of formula (XII) via [2+2] cycloaddition reaction with a ketene such as dichloroketene.

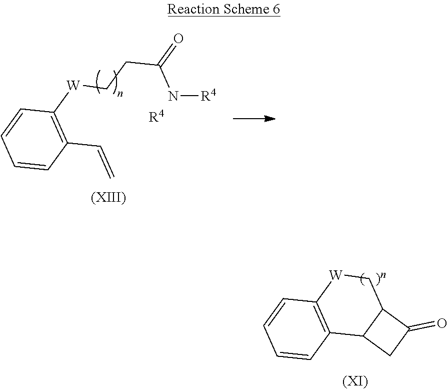

Alternatively, compounds of formula (XI) can be prepared from a compound of formula (XIII) via [2+2]cycloaddition reaction with a keteniminium salt using a base such as sym-collidine or 2-halogeno pyridine (e.g. 2-fluoropyridine) and triflic anhydride.

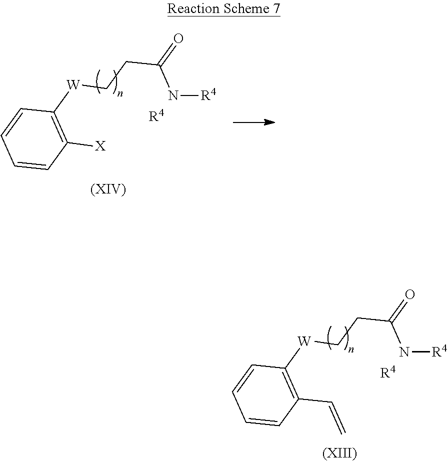

Compounds of formula (XIII) can be prepared from a compound of formula (XIV), wherein R⁴ are C₁-C₄ alkyl group, C₃-C₆ alkenyl group or are joined to form a 5-7 membered cycloalkyl ring; X is Br, Cl or I, and a vinyl metal derivative, wherein [M] can be a boron or a tin derivatives, in presence of a suitable catalyst/ligand system, often palladium (0) complex.

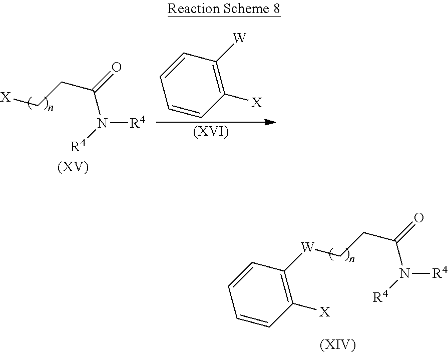

Compounds of formula (XIV) can be prepared from known compounds of formula (XV) and (XVI) wherein X is Br or I, using a base such as triethylamine amine or sodium hydride

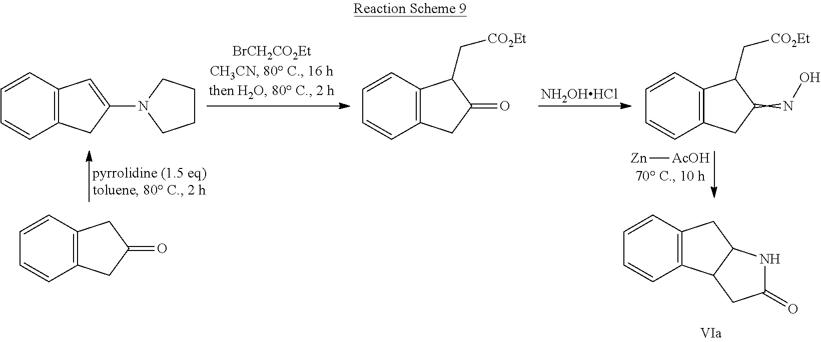

Alternatively compounds of formula VI (W═CH₂, n=0) can be prepared from 2-indanones following procedures known in the art (Tetrahedron Lett. 1971, 29, 2787-2790).

### PREPARATION EXAMPLES

The following Examples serve to illustrate the invention.

### Compound Synthesis and Characterisation

The following abbreviations are used throughout this section: s=singlet; bs=broad singlet; d=doublet; dd=double doublet; dt=double triplet; bd=broad doublet; t=triplet; td=triplet doublet; bt=broad triplet; tt=triple triplet; q=quartet; m=multiplet; Me=methyl; Et=ethyl; Pr=propyl; Bu=butyl; DME=1,2-dimethoxyethane; THE=tetrahydrofuran; M.p.=melting point; RT=retention time, MH⁺=molecular cation (i.e. measured molecular weight).

The following HPLC-MS methods were used for the analysis of the compounds:

Method A: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-1.2 min 100% B; 1.2-1.50 min 100% B.

Method B: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-2.7 min 100% B; 2.7-3.0 min 100% B.

### Example P1: Preparation of 2,2-dichloro-7,7a-dihydro-2aH-cyclobuta[a]inden-1-one (IX-a)

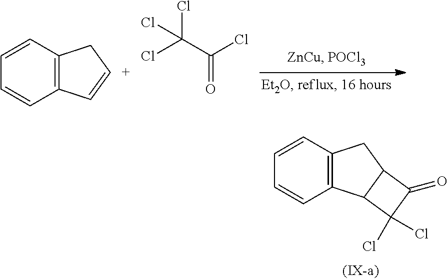 [SMILES: [C+]1=Cc2ccccc2C1 ; O=C(Cl)C(Cl)(Cl)Cl ; O=C1C2Cc3ccccc3C2C1(Cl)Cl]

To a flask under argon was added dry diethyl ether (450 mL), indene (250 mmol, 30.1 mL) (450 mL), and cuprouszinc (751 mmol, 96.9 g). To this suspension was added a solution of trichloroacetylchloride (501 mmol, 56.5 mL) and phosphorus oxychloride (275 mmol, 25.9 mL) in diethyl ether (150 mL). After complete addition, the suspension was heated at reflux for 16 hours. The reaction mixture was then filtered through a Celite® pad which was washed with diethyl ether. The filtrate was washed with water, saturated aqueous NaHCO₃ solution and brine. The organic phase was then dried over sodium sulfate, filtered, concentrated under reduced pressure and the obtained crude residue was finally purify by column chromatography on silica gel affording compound of formula (IX-a) as off-white solid in 97% yield (242 mmol, 55.0 g). ¹H NMR (400 MHz, CDCl₃) δ ppm 7.47 (m, 1H), 7.25-7.40 (m, 3H), 4.48-4.57 (m, 2H), 3.43 (d, 1H), 3.22 (dd, 1H).

Using a similar procedure, (IX-b) and (IX-d) were prepared

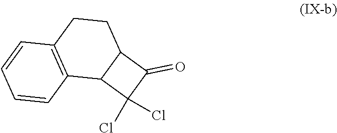 [SMILES: O=C1C2CCc3ccccc3C2C1(Cl)Cl]

1,1-dichloro-2a,3,4,8b-tetrahydrocyclobuta[a]naphthalen-2-one; LCMS (Method A): RT 1.09 min; ES+ 239 (M−H⁺);

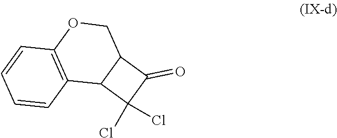 [SMILES: O=C1C2COc3ccccc3C2C1(Cl)Cl]

1,1-dichloro-3,8b-dihydro-2aH-cyclobuta[c]chromen-2-one; ¹H NMR (400 MHz, CDCl₃) δ ppm 7.34-7.24 (m, 2H), 7.09 (m, 1H), 6.96 (m, 1H), 4.62 (dd, 1H), 4.34 (m, 1H), 4.25 (d, 1H), 3.89 (dd, 1H).

### Example P2: Preparation of 1,1-dichloro-3,3a,4,8b-tetrahydroindeno[2,1-b]pyrrol-2-one (X-a)

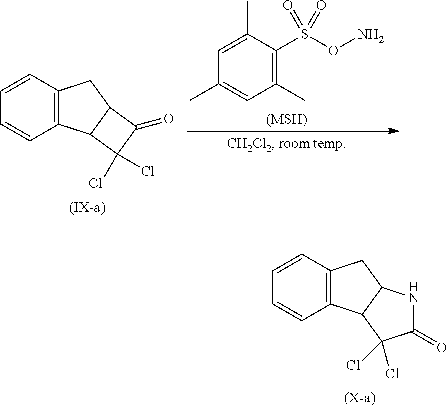

To a solution of compound of formula (IX-a) (44 mmol, 10.0 g) in dichloromethane (290 mL) at room temperature was added known Mesityl Sulfonyl Hydroxylamine (MSH, 46 mmol, 9.9 g) (refer Angew. Chem. Int. Ed. 2011, 50, 4127-4132 for preparation of MSH) and a spun of Na₂SO₄. The resulting mixture was stirred at room temperature for 7 days (additional 0.5 equivalent of freshly prepared MSH was added after 4 days). The suspension was then filtered on Celite® and the filter cake was washed with dichloromethane. The filtrate was concentrated under reduced pressure (crude residue was kept in a minimum of solvent due to potential presence of residual MSH) and the crude residue was purified by flash chromatography on silica gel. Compound of formula (X-a) was isolated as a white solid in 70% yield (31 mmol, 7.5 g). LCMS (Method A): RT 0.83 min; ES+ 243 (M+H+); ¹H NMR (400 MHz, CDCl₃) δ ppm 7.60 (d, 1H), 7.40 (bs, 1H), 7.13-7.28 (m, 3H), 4.55 (td, 1H), 4.42 (d, 1H), 3.16 (dd, 1H), 2.98 (dd, 1H).

Using a similar procedure, (X-b) and (X-d) were prepared

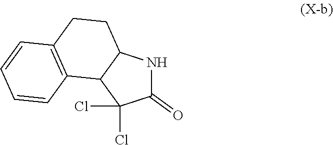 [SMILES: O=C1NC2CCc3ccccc3C2C1(Cl)Cl]

1,1-dichloro-3a,4,5,9b-tetrahydro-3H-benzo[e]indol-2-one; LCMS (Method A): RT 0.85 min; ES+256 (M+H⁺).

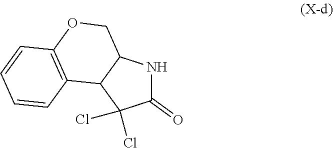 [SMILES: O=C1NC2COc3ccccc3C2C1(Cl)Cl]

1,1-dichloro-3,3a,4,9b-tetrahydrochromeno[3,4-b]pyrrol-2-one; LCMS (Method A): RT 0.81 min; ES+ 258 (M+H⁺);

### Example P3-1: Preparation of 3,3a,4,8b-tetrahydro-1H-indeno[2,1-b]pyrrol-2-one (VIII-a)

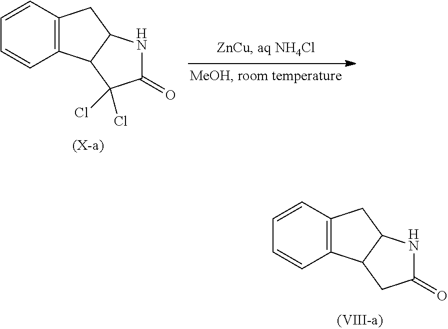 [SMILES: O=C1NC2Cc3ccccc3C2C1(Cl)Cl ; O=C1CC2c3ccccc3CC2N1]

Compound of formula (X-a, 8.3 mmol, 2.0 g) was dissolved in a saturated solution of ammonium chloride (41.5 mmol, 2.2 g) in methanol (80 mL) then cuprouszinc (33.2 mmol, 4.2 g) was added and the resulting suspension was stirred at room temperature for 16 hours. The reaction mixture was then filtered on Celite, the filter cake washed with MeOH and the filtrate was evaporated under reduced pressure to afford 3.5 g of a white residue which was suspended in EtOAc and washed with water several times. The combined water was then re-extracted with EtOAc. The combined organic fractions were then washed with brine, dried over Na₂SO₄, filtered and concentrated under reduced pressure to afford compound of formula (VIII-a) as a white solid in 99% yield (8.2 mmol, 1.4 g). LCMS (Method A): RT 0.63 min; ES+ 174 (M+H+); ¹H NMR (400 MHz, CDCl₃) δ ppm 7.22-7.32 (m, 4H), 6.06 (bs, 1H), 4.53 (t, 1H), 3.98 (m, 1H), 3.27 (dd, 1H), 2.99 (d, 1H), 2.90 (dd, 1H), 2.53 (d, 1H).

Using a similar procedure, (VIII-b) and (VIII-d) were prepared

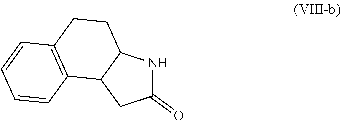 [SMILES: O=C1CC2c3ccccc3CCC2N1]

1,3,3a,4,5,9b-hexahydrobenzo[e]indol-2-one; LCMS (Method A): RT 0.70 min; ES+ 188 (M+H⁺);

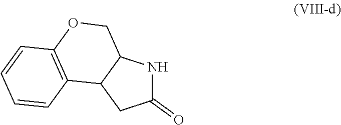 [SMILES: O=C1CC2c3ccccc3OCC2N1]

3,3a,4,9b-tetrahydro-1H-chromeno[3,4-b]pyrrol-2-one; LCMS (Method A): RT 0.60 min; ES+190 (M+H⁺);

### Example P4: Preparation of 1,3,3a,8b-tetrahydrobenzofuro[2,3-b]pyrrol-2-one (VIII-c)

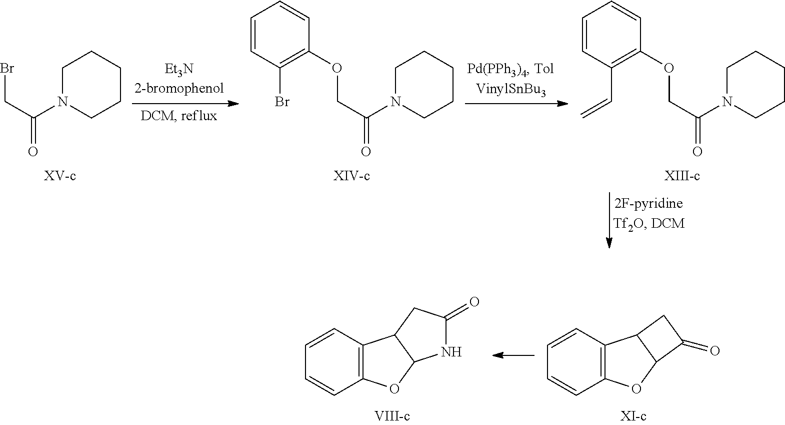 [SMILES: O=C(CBr)N1CCCCC1 ; O=C(COc1ccccc1Br)N1CCCCC1 ; C=Cc1ccccc1OCC(=O)N1CCCCC1 ; O=C1CC2c3ccccc3OC2N1 ; O=C1CC2c3ccccc3OC12]

To a solution of compound (XV-c, 1.0 g, 4.8 mmol) and K₂CO₃ (2.0 eq, 9.7 mmol) in DMF (9.7 mL) was added at 0° C. 2-bromophenol (1.2 equiv., 5.8 mmol). The reaction was then heated under Argon atmosphere at 60° C. for 1 h30. The reaction mixture was then partitioned between water and CH₂Cl₂ and the phases separated. The aqueous phase was extracted with a further portion of CH₂Cl₂ and the combined organic layers were washed with water, dried over magnesium sulphate and concentrated under vacuum. The resulting crude residue was purified by flash chromatography on SiO2 affording compound (XIV-c) as a colorless oil crystallizing on standing in 91% yield (1.3 g). LCMS (Method A): RT 0.95 min; ES⁺ 300 (M+H⁺).

A solution compound (XIV-c) (1 g, 3.35 mmol) in toluene (13 mL) was degassed with Argon for 30 min and vinyl stannane (1.3 equiv., 4.36 mmol) followed by Pd(PPh₃)₄ (0.33 mmol, 99.8 mass %) were added, and the reaction mixture was heated to 100° C. and stirred for 16 hours. The reaction mixture was then cooled, concentrated under vacuum and purified by flash chromatography on SiO2 affording compound (XIII-c) as a colorless oil in 91% yield (0.75 g, 3.06 mmol). LCMS (Method A): RT 0.97 min; ES+ 247 (M+H⁺).

To a stirred solution of compound (XIII-c, 11 g, 44.8 mmol) in CH₂Cl₂ (179 mL) was added sequentially at room temperature 2-fluoropyridine (1.3 eq, 58.3 mmol, 5.1 mL) and Tf₂O (1.2 eq, 53.8 mmol, 9.05 mL) dropwise and the resulting reaction mixture was stirred for 14 hours. The solvent was then removed under vacuum and the crude residue was diluted in carbon tetrachloride (100 mL) and water (100 mL) and the biphasic mixture was stirred at 65° C. for additional 16 hours. Reaction mixture was extracted with CH₂Cl₂, dried over sodium sulfate and concentrated under reduce pressure. Purification by flash chromatography on SiO₂ afforded compound (XI-c) in 77% yield (5.5 g, 34 mmol). ¹H NMR (400 MHz, CDCl₃) δ ppm 7.32 (m, 1H), 7.22 (m, 1H), 6.98 (td, 1H), 6.91 (m, 1H), 5.75 (dt, 1H), 4.25 (td, 1H), 3.67 (ddd, 1H), 3.11 (dt, 1H).

To a solution of compound (XI-c) (2 g, 12.5 mmol) in CH₂Cl₂ (83 mL) at room temperature was added aqHCl solution (5 eq, 2M, 31.2 mL) and MSH (Mesityl Sulfonyl Hydroxylamine) (1.5 eq, 4.03 g, 18.7 mmol) The reaction stirred for 16 hours at room temperature and the organic layer was then washed with a saturated solution of NaHCO₃. The organic layer was then dried over sodium sulfate and concentrated under vacuum. Compound (VIII-c) was obtained as a colorless oil crystallizing on standing in 91% yield (2.0 g, 11.4 mmol) and used without further purification. LCMS (Method A): RT 0.58 min; ES+ 176 (M+H⁺).

### Example P5: Preparation of tert-butyl 2-oxo-1,3a,4,8b-tetrahydroindeno[2,1-b]pyrrole-3-carboxylate (VI)

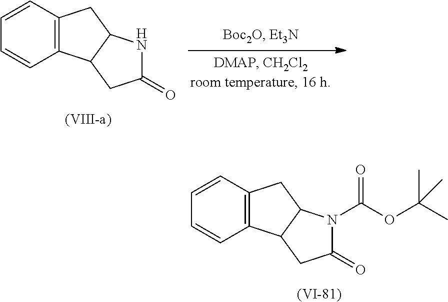

Compound of formula (VIII-a, 8.3 mmol, 1.4 g) was dissolved in CH₂Cl₂ (40 mL) and tert-butoxycarbonyl tert-butyl carbonate (10.0 mmol, 2.2 g), triethylamine (16.6 mmol, 2.3 mL), N,N-dimethylpyridin-4-amine (0.41 mmol, 0.05 g) was then added to the solution. The reaction mixture was stirred for 16 hours at room temperature. The medium was then washed with HCl (1M) and the aqueous layer was extracted with CH₂Cl₂. The combined organic layers were washed with a solution of NaHCO₃, dried over Na₂SO₄, filtered and evaporated to afford compound of formula (VI-81) in quantitative yield (8.4 mmol, 2.3 gram). ¹H NMR (400 MHz, CDCl₃) δ ppm 7.10-7.22 (m, 4H), 4.80 (td, 1H), 3.77 (m, 1H), 3.38 (dd, 1H), 3.11 (dd, 1H), 2.97 (dd, 1H), 2.60 (dd, 1H), 1.49 (s, 9H).

TABLE 2
Compounds of formula (VI-9, VI-10, VI-12, VI-49 and VI-83) were prepared from VIII-a,
VIII-b and VIII-c using procedures described in WO2012/080115 (Rₜ = Retention time)

| Cpd No. | Structure | Name | LCMS or ¹H NMR |
|---|---|---|---|
| VI-9 | 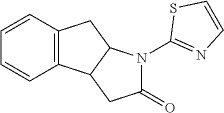 [SMILES: O=C1CC2c3ccccc3CC2N1c1nccs1] | 3-thiazol-2-yl-1,3a,4,8b- tetrahydroindeno[2,1-b]pyrrol-2- one | Rₜ = 0.92 min (Method A); ES+ 257 (M + H⁺) |
| VI-10 | 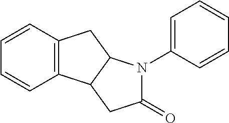 [SMILES: O=C1CC2c3ccccc3CC2N1c1ccccc1] | 3-phenyl-1,3a,4,8b- tetrahydroindeno[2,1-b]pyrrol-2- one | Rₜ = 0.95 min (Method A); ES+ 250 (M + H⁺) |
| VI-12 | 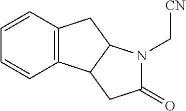 [SMILES: N#CCN1C(=O)CC2c3ccccc3CC21] | 2-(2-oxo-1,3a,4,8b- tetrahydroindeno[2,1-b]pyrrol-3- yl)acetonitrile | Rₜ = 0.73 min (Method A); ES+ 213 (M + H⁺) |
| VI-49 | 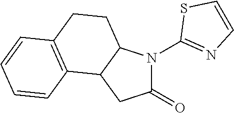 [SMILES: O=C1CC2c3ccccc3CCC2N1c1nccs1] | 3-thiazol-2-yl-3a,4,5,9b- tetrahydro-1H-benzo[e]indol-2-one | Rₜ = 0.97 min (Method A); ES+ 271 (M + H⁺) |
| VI-83 | 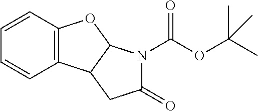 [SMILES: CC(C)(C)OC(=O)N1C(=O)CC2c3ccccc3OC21] | tert-butyl 2-oxo-3a,8b-dihydro-1H- benzofuro[2,3-b]pyrrole-3- carboxylate | 7.25-7.14 (m, 2H), 6.95 (m 1H), 6.87 (d, 1H), 6.53 (d, 1H), 4.04 (m, 1H), 3.07 (dd, 1H), 2.71 (dd, 1H), 1.59 (s, 9H). |

### Example P6: Preparation of tert-butyl (1E)-1-(hydroxymethylene)-2-oxo-4,8b-dihydro-3aH-indeno[2,1-b]pyrrole-3-carboxylate (IV-81)

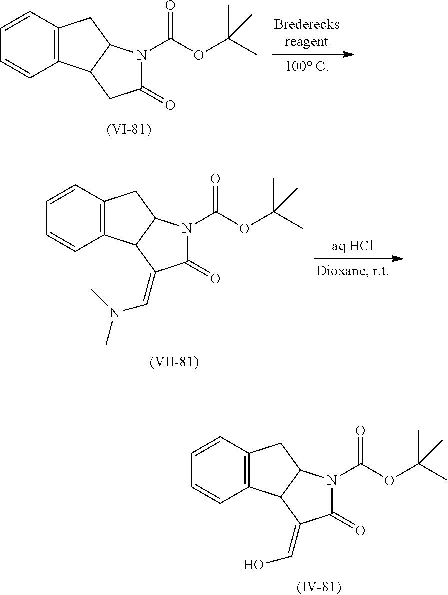

Compound of formula (VI-81) (11.0 mmol, 3.3 g) was treated with Bredereck's reagent (tert butoxybis(dimethylamino)methane) (34 mmol, 6.7 g) under argon and the reaction mixture was heated to 100° C. (brown solution) for 1 h45 min. After cooling to room temperature, the reaction mixture was diluted with ethyl acetate (150 ml) and washed with water follow by brine, dried over Na₂SO₄ and the solvent was evaporated under reduced pressure. The crude reaction residue was then treated with pentane and the resulting solid was filtered off affording compound of formula (VII-81) in 90% yield (10.3 mmol, 3.4 g). Compound of formula (VII-81) (7.8 mmol, 2.5 g) was then dissolved in 1,4-dioxane (40.0 mL) and aqueous hydrochloric acid solution (1M, 15.5 mL) and the resulting reaction mixture was stirred for 35 minutes at room temperature. Brine was added and extraction was done with ethyl acetate. The combined organic fractions were dried over sodium sulfate, the solvents, evaporated and the resulting crude residue was purified by flash chromatography affording compound of formula (IV-81) in 96% yield (7.4 mmol, 2.2 g). LCMS (Method A): RT 0.95 min; ES− 300 (M−H+); ¹H NMR (400 MHz, CDCl₃) δ ppm 11.1 (bs, 1H), 7.14-7.34 (m, 4H), 4.95 (td, 1H), 4.38 (d, 1H), 3.56 (dd, 1H), 3.20 (dd, 1H), 1.61 (s, 9H).

TABLE 3
Compounds of formula (IV-12, IV-49 and IV-83) were prepared from VI-12, VI-49 and VI-
83 using same procedure as described for compound (IV-81). (Rₜ = Retention time)

| Cpd | | | |
|---|---|---|---|
| No. | Structure | Name | LCMS |
| IV-12 | 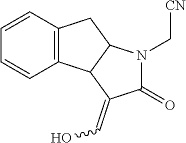 [SMILES: N#CCN1C(=O)C(=CO)C2c3ccccc3CC21] | 2-[(1E/Z)-1- (hydroxymethylene)-2- oxo-4,8b-dihydro-3aH- indeno[2,1-b]pyrrol-3- yl]acetonitrile | Rₜ = 0.73 min (Method A); ES+ 241 (M + H⁺) |
| IV-49 | 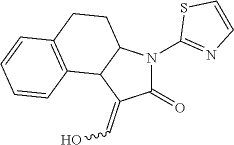 [SMILES: O=C1C(=CO)C2c3ccccc3CCC2N1c1nccs1] | (1E/Z)-1- (hydroxymethylene)-3- thiazol-2-yl-3a,4,5,9b- tetrahydrobenzo[e]indol- 2-one | Rₜ = 0.92 min (Method A); ES+ 299 (M + H⁺) |
| IV-83 | 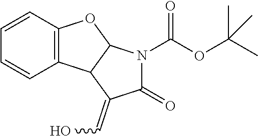 [SMILES: CC(C)(C)OC(=O)N1C(=O)C(=CO)C2c3ccccc3OC21] | tert-butyl (1E)-1- (hydroxymethylene)-2- oxo-3a,8b- dihydrobenzofuro[2,3- b]pyrrole-3-carboxylate | Rₜ = 0.90 min (Method A); ES− 302 (M − H⁺) |

### Example P7: Preparation of (E/Z)-1-(hydroxymethylene)-3-thiazol-2-yl-4,8b-dihydro-3aH-indeno[2,1-b]pyrrol-2-one (IV-9)

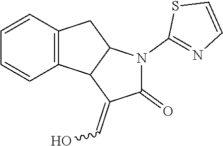 [SMILES: O=C1C(=CO)C2c3ccccc3CC2N1c1nccs1]

To a solution under argon of compound (VI-9) (4.00 g, 15.6 mmol) in THE (78.0 mL) was added dropwise at −78° C. lithium bis(trimethylsilyl)amide (1M in THF, 23 mL, 1.5 eq) solution. The resulting solution was then warmed to −50° C. and stirred for 30 min. Ethyl formate (3.88 mL, 46.8 mmol) was added and the reaction was allowed to warm slowly to room temperature. Water was added to the reaction mixture and pH was adjusted to 4. The aqueous layer was extracted with EtOAc and the combined organic fractions were dried over sodium sulfate, filtered and concentrated under reduced pressure. The resulting crude residue was stirred in ethylacetate and then filtered off affording compound (IV-9) as a beige solid in 79% yield (3.5 g, 12 mmol). LCMS (Method A): RT 0.88 min; ES+285 (M+H+).

Using a similar procedure, (1E)-1-(hydroxymethylene)-3-phenyl-4,8b-dihydro-3aH-indeno[2,1-b]pyrrol-2-one (IV-10) was prepared

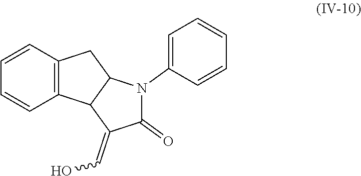 [SMILES: O=C1C(=CO)C2c3ccccc3CC2N1c1ccccc1]

LCMS (Method A): RT 0.94 min; ES+ 278 (M+H+)

### Example P8: Preparation of tert-butyl (1E)-1-[(4-methyl-5-oxo-2H-furan-2-yl)oxymethylene]-2-oxo-4,8b-dihydro-3aH-indeno[2,1-b]pyrrole-3-carboxylate (I-81)

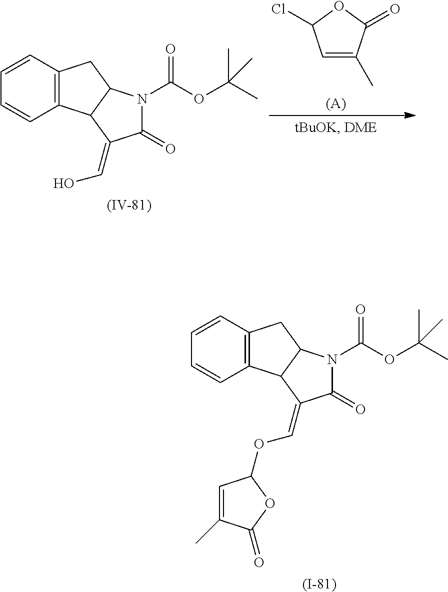

Compound of formula (IV-81) (0.61 mmol) was dissolved in anhydrous 1,2-dimethoxyethane (4 mL), the resulting solution cooled to 0° C. and tBuOK (0.08 g, 0.73 mmol) was then added. After 10 minutes at 0° C., known compound of formula (A) (0.74 mmol) was added as a solution in 1 mL of DME. The reaction mixture was then slowly warmed to room temperature. After 16 hours, a saturated aqueous NH₄Cl solution was added and the reaction mixture was extracted with ethyl acetate. The combined organic extracts were washed with brine, dried over sodium sulfate and concentrated under vacuum. The crude reaction residue was purified by flash chromatography on silica gel affording compound of formula (I-81) as a colorless oil and as a mixture of diastereoisomers in 94% yield (0.57 mmol). ¹H NMR (400 MHz, CDCl₃) δ ppm (data given for the two diastereoisomers) 7.50 (d, 1H), 7.45 (d, 1H), 7.35 (m, 2H), 7.24-7.15 (m, 6H), 7.02 (m, 1H), 6.99 (m, 1H), 6.22 (m, 2H), 4.82 (m, 2H), 4.57 (m, 2H), 3.51 (m, 1H), 3.47 (m, 1H), 3.20 (m, 1H), 3.15 (m, 1H), 2.06 (m, 6H), 1.57 (s, 9H), 1.56 (s, 9H).

TABLE 4
The following compounds of formula (I) were prepared using a similar procedure to the
one described for compound (I-81) using known compound (A) or (B). (Rₜ = Retention time)

| Cpd | | | |
|---|---|---|---|
| No. | Structure | Name | LCMS or NMR |
| I-82 | 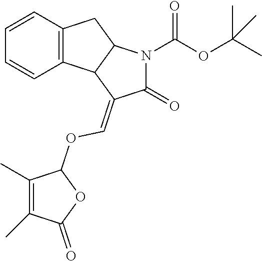 [SMILES: CC1=C(C)C(O/C=C2/C(=O)N(C(=O)OC(C)(C)C)C3Cc4ccccc4C23)OC1=O] | tert-butyl (1E)-1-[(3,4- dimethyl-5-oxo-2H- furan-2-yl) oxymethylene]-2-oxo- 4,8b-dihydro-3aH- indeno[2,1-b]pyrrole-3- carboxylate | ¹H NMR (400 MHz, CDCl₃) δ ppm (data given for the two diastereoisomers) 7.48 (d, 1H), 7.40 (d, 1H), 7.36 (m, 2H), 7.23- 7.14 (m, 6H), 6.03 (s, 2H), 4.83 (m, 2H), 4.57 (m, H), 3.52 (m, 1H), 3.48 (m, 1H), 3.20 (m, 1H), 3.15 (m, 1H), 2.10 (s, 3H), 2.04 (s, 3H), 1.93 (s, 6H), 1.57 (s, 9H), 1.56 (s, 9H) |
| I-12-E | 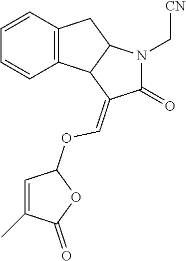 [SMILES: CC1=CC(O/C=C2/C(=O)N(CC#N)C3Cc4ccccc4C23)OC1=O] | 2-[(1E)-1-[(4-methyl-5- oxo-2H-furan-2- yl)oxymethylene]-2-oxo- 4,8b-dihydro-3aH- indeno[2,1-b]pyrrol-3- yl]acetonitrile | Rₜ = 0.85 min (Method A); ES+ 337 (M + H⁺) |
| I-12-Z | 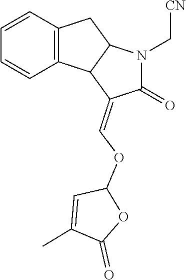 [SMILES: CC1=CC(O/C=C2\C(=O)N(CC#N)C3Cc4ccccc4C23)OC1=O] | 2-[(1Z)-1-[(4-methyl-5- oxo-2H-furan-2- yl)oxymethylene]-2-oxo- 4,8b-dihydro-3aH- indeno[2,1-b]pyrrol-3- yl]acetonitrile | Rₜ = 0.88 min (Method A); ES+ 337 (M + H⁺) |
| I-16-E | 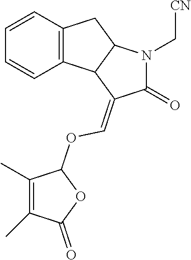 [SMILES: CC1=C(C)C(O/C=C2/C(=O)N(CC#N)C3Cc4ccccc4C23)OC1=O] | 2-[(1E)-1-[(3,4-dimethyl- 5-oxo-2H-furan-2- yl)oxymethylene]-2-oxo- 4,8b-dihydro-3aH- indeno[2,1-b]pyrrol-3- yl]acetonitrile | Rₜ = 0.92 min (Method A); ES+ 351 (M + H⁺) |
| I-16-Z | 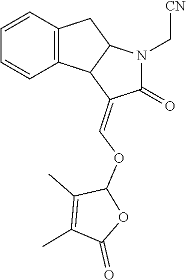 [SMILES: CC1=C(C)C(O/C=C2\C(=O)N(CC#N)C3Cc4ccccc4C23)OC1=O] | 2-[(1Z)-1-[(3,4-dimethyl- 5-oxo-2H-furan-2- yl)oxymethylene]-2-oxo- 4,8b-dihydro-3aH- indeno[2,1-b]pyrrol-3- yl]acetonitrile | Rₜ = 0.88 min (Method A); ES+ 351 (M + H⁺) |
| I-10 | 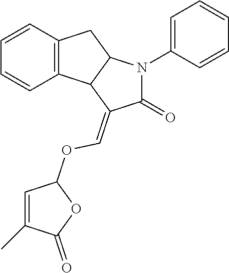 [SMILES: CC1=CC(O/C=C2/C(=O)N(c3ccccc3)C3Cc4ccccc4C23)OC1=O] | (1E)-1-[(4-methyl-5-oxo- 2H-furan-2- yl)oxymethylene]-3- phenyl-4,8b-dihydro- 3aH-indeno[2,1-b]pyrrol- 2-one | Rₜ = 1.64/1.65 min (Method A); ES+ 374 (M + H⁺) |
| I-14 | 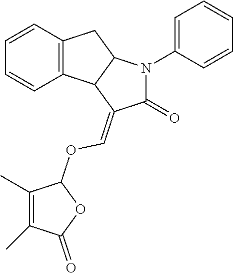 [SMILES: CC1=C(C)C(O/C=C2/C(=O)N(c3ccccc3)C3Cc4ccccc4C23)OC1=O] | (1E)-1-[(3,4-dimethyl-5- oxo-2H-furan-2- yl)oxymethylene]-3- phenyl-4,8b-dihydro- 3aH-indeno[2,1-b]pyrrol- 2-one | Rₜ = 1.07/1.08 min (Method A); ES+ 388 (M + H⁺) |
| I-9 | 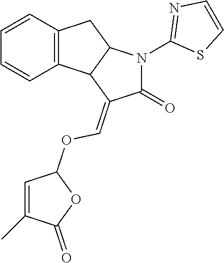 [SMILES: CC1=CC(O/C=C2/C(=O)N(c3nccs3)C3Cc4ccccc4C23)OC1=O] | (1E)-1-[(4-methyl-5-oxo- 2H-furan-2- yl)oxymethylene]-3- thiazol-2-yl-4,8b- dihydro-3aH-indeno[2,1- b]pyrrol-2-one | Rₜ = 1.56/1.57 min (Method A); ES+ 381 (M + H⁺) |
| I-13 | 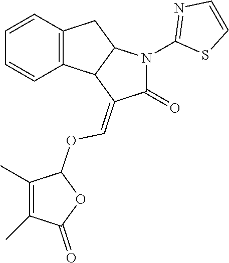 [SMILES: CC1=C(C)C(O/C=C2/C(=O)N(c3nccs3)C3Cc4ccccc4C23)OC1=O] | (1E)-1-[(3,4-dimethyl-5- oxo-2H-furan-2- yl)oxymethylene]-3- thiazol-2-yl-4,8b- dihydro-3aH-indeno[2,1- b]pyrrol-2-one | Rₜ = 1.03/1.04 min (Method A); ES+ 395 (M + H⁺) |
| I-49 | 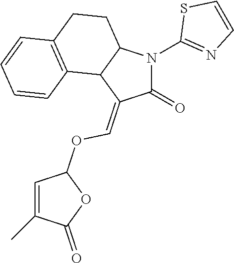 [SMILES: CC1=CC(O/C=C2/C(=O)N(c3nccs3)C3CCc4ccccc4C23)OC1=O] | (1E)-1-[(4-methyl-5-oxo- 2H-furan-2- yl)oxymethylene]-3- thiazol-2-yl-3a,4,5,9b- tetrahydrobenzo[e]indol- 2-one | Rₜ = 1.00/1.03 min (Method A); ES+ 395 (M + H⁺) |
| I-53 | 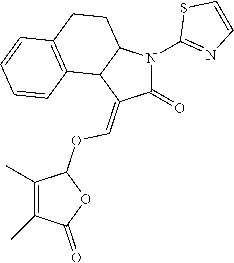 [SMILES: CC1=C(C)C(O/C=C2/C(=O)N(c3nccs3)C3CCc4ccccc4C23)OC1=O] | (1E)-1-[(3,4-dimethyl-5- oxo-2H-furan-2- yl)oxymethylene]-3- thiazol-2-yl-3a,4,5,9b- tetrahydrobenzo[e]indol- 2-one | Rₜ = 1.04/1.06 min (Method A); ES+ 409 (M + H⁺) |
| I-83 | 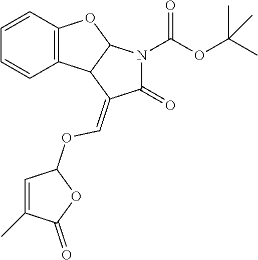 [SMILES: CC1=CC(O/C=C2/C(=O)N(C(=O)OC(C)(C)C)C3Oc4ccccc4C23)OC1=O] | tert-butyl (1E)-1-[(4- methyl-5-oxo-2H-furan- 2-yl)oxymethylene]-2- oxo-3a,8b- dihydrobenzofuro[2,3- b]pyrrole-3-carboxylate | Rₜ = 1.02/1.03 min (Method A); ES+ 300 (M + H⁺ − Boc) |
| I-84 | 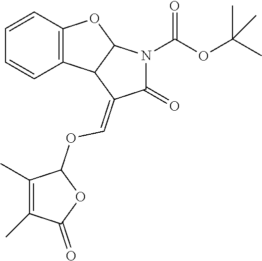 [SMILES: CC1=C(C)C(O/C=C2/C(=O)N(C(=O)OC(C)(C)C)C3Oc4ccccc4C23)OC1=O] | tert-butyl (1E)-1-[(3,4- dimethyl-5-oxo-2H- furan-2- yl)oxymethylene]-2-oxo- 3a,8b- dihydrobenzofuro[2,3- b]pyrrole-3-carboxylate | Rₜ = 1.03/1.04 min (Method A); ES+ 414 (M + H⁺) |

### Example P9: Preparation of (1E)-1-[(4-methyl-5-oxo-2H-furan-2-yl)oxymethylene]-3,3a,4,8b-tetrahydroindeno[2,1-b]pyrrol-2-one (V-a1)

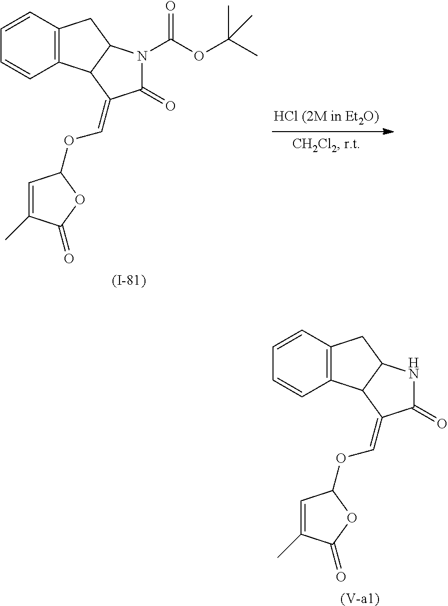

Compound of formula (I-81) (1.610 mmol) was dissolved in CH₂Cl₂ (12.88 mL) and HCl (2.0 M in Et₂O, 2.416 mL) was added dropwise. The reaction mixture was then stirred for 1.5 h at r.t., neutralized with aqNaHCO₃ and extracted with CH₂Cl₂. The organic layers were combined, dried over sodium sulfate and concentrated under vacuum affording compound (V-a1) in 88% yield (1.413 mmol). LCMS (Method A): RT 0.81/0.82 min; ES+ 298 (M+H⁺).

Using a similar procedure, (V-a2) and (V-c2) were prepared using TFA instead of HCl

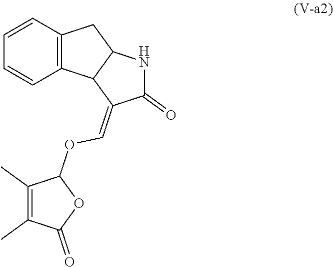 [SMILES: CC1=C(C)C(O/C=C2/C(=O)NC3Cc4ccccc4C23)OC1=O]

(1E)-1-[(3,4-dimethyl-5-oxo-2H-furan-2-yl)oxymethylene]-3,3a,4,8b-tetrahydroindeno[2,1 b]pyrrol-2-one; LCMS (Method A): RT 0.85/0.86 min; ES+ 312 (M−H⁺);

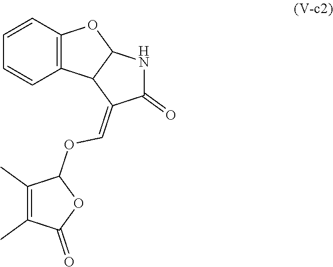 [SMILES: CC1=C(C)C(O/C=C2/C(=O)NC3Oc4ccccc4C23)OC1=O]

(1E)-1-[(3,4-dimethyl-5-oxo-2H-furan-2-yl)oxymethylene]-3a,8b-dihydro-3H-benzofuro[2,3-b]pyrrol-2-one; LCMS (Method A): RT 0.80/0.82 min; ES+ 314 (M−H⁺);

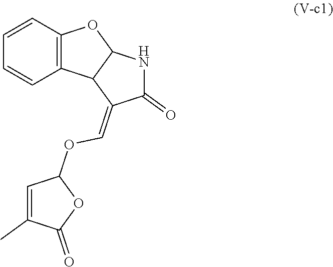 [SMILES: CC1=CC(O/C=C2/C(=O)NC3Oc4ccccc4C23)OC1=O]

(1E)-1-[(4-methyl-5-oxo-2H-furan-2-yl)oxymethylene]-3a,8b-dihydro-3H-benzofuro[2,3-b]pyrrol-2-one; LCMS (Method A): RT 0.76/0.78 min; ES+ 322 (M+Na⁺);

### Example P10: Preparation of (1E)-3-acetyl-1-[(4-methyl-5-oxo-2H-furan-2-yl)oxymethylene]-4,8b-dihydro-3aH-indeno[2,1-b]pyrrol-2-one (I-1)

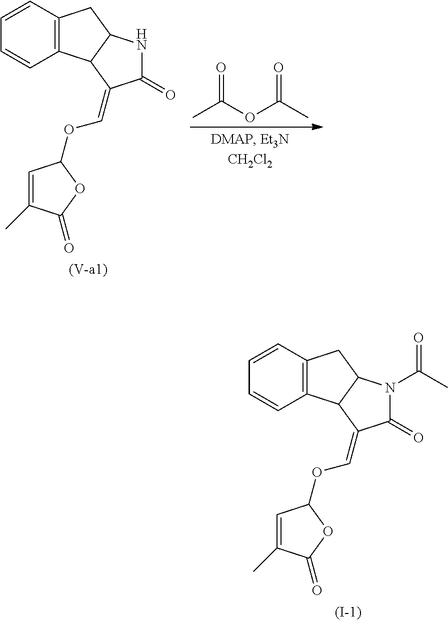

To a degassed solution of compound (Va-1, 0.4 g, 1.345 mmol) in dichloromethane (12.1 mL) was added dimethylamino pyridine (DMAP) (0.008 g, 0.067 mmol) and Et₃N (0.758 mL, 5.382 mmol) followed by dropwise addition of acetic anhydride (0.39 mL, 4.03 mmol) at r.t. The reaction mixture was then stirred overnight, poured into sat aqNH₄Cl solution and diluted with ethyl acetate. The phases were separated and the organic layer was dried over sodium sulfate and concentrated under vacuum. The crude reaction mixture was purified by flash chromatography affording compound (I-1) in 75% yield 0.34 g, 1.00 mmol). ¹H NMR (400 MHz, CDCl₃) δ ppm (data given for the two diastereoisomers) 7.53 (d, 1H), 7.50 (d, 1H), 7.41-7.34 (m, 2H), 7.24-7.15 (m, 6H), 7.04 (m, 1H), 7.01 (m, 1H), 6.25 (bs, 2H), 4.93 (m, 2H), 4.61 (m, 1H), 4.59 (m, 1H), 3.59 (m, 1H), 3.54 (m, 1H), 3.15 (m, 1H), 3.10 (m, 1H), 2.54 (s, 3H), 2.53 (s, 3H), 2.08 (m, 6H). LCMS (Method A): RT 1.42/1.43 min; ES+ 340 (M+H⁺).

TABLE 5
The following compounds of formula (I) were prepared via a similar procedure using
specific anhydride or acyl chloride (Rₜ = Retention time)

| Cpd | | | |
|---|---|---|---|
| No. | Structure | Name | LCMS or NMR |
| I-5 | 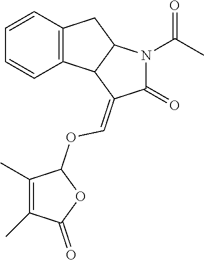 [SMILES: CC(=O)N1C(=O)/C(=C/OC2OC(=O)C(C)=C2C)C2c3ccccc3CC21] | (1E)-3-acetyl-1-[(3,4- dimethyl-5-oxo-2H-furan-2- yl)oxymethylene]-4,8b- dihydro-3aH-indeno[2,1- b]pyrrol-2-one | Rₜ = 0.97/0.98 min (Method A); ES+ 354 (M + H⁺) |
| I-2 | 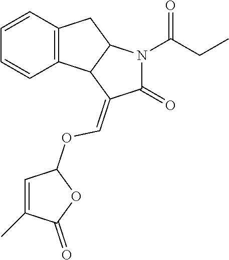 [SMILES: CCC(=O)N1C(=O)/C(=C/OC2C=C(C)C(=O)O2)C2c3ccccc3CC21] | (1E)-1-[(4-methyl-5-oxo-2H- furan-2-yl)oxymethylene]-3- propanoyl-4,8b-dihydro- 3aH-indeno[2,1-b]pyrrol-2- one | Rₜ = 1.00/1.01 min (Method A); ES+ 354 (M + H⁺) |
| I-6 | 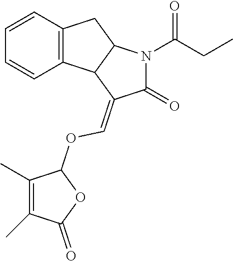 [SMILES: CCC(=O)N1C(=O)/C(=C/OC2OC(=O)C(C)=C2C)C2c3ccccc3CC21] | (1E)-1-[(3,4-dimethyl-5-oxo- 2H-furan-2- yl)oxymethylene]-3- propanoyl-4,8b-dihydro- 3aH-indeno[2,1-b]pyrrol-2- one | Rₜ = 1.03/1.04 min (Method A); ES+ 368 (M + H⁺) |
| I-3 | 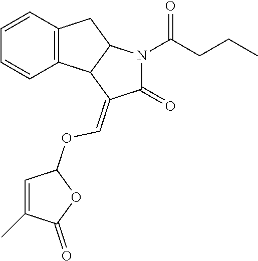 [SMILES: CCCC(=O)N1C(=O)/C(=C/OC2C=C(C)C(=O)O2)C2c3ccccc3CC21] | (1E)-3-butanoyl-1-[(4- methyl-5-oxo-2H-furan-2- yl)oxymethylene]-4,8b- dihydro-3aH-indeno[2,1- b]pyrrol-2-one | Rₜ = 1.05/1.06 min (Method A); ES+ 368 (M + H⁺) |
| I-7 | 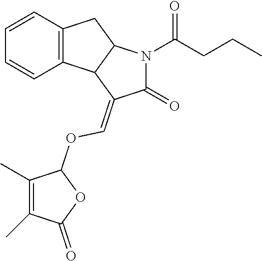 [SMILES: CCCC(=O)N1C(=O)/C(=C/OC2OC(=O)C(C)=C2C)C2c3ccccc3CC21] | (1E)-3-butanoyl-1-[(3,4- dimethyl-5-oxo-2H-furan-2- yl)oxymethylene]-4,8b- dihydro-3aH-indeno[2,1- b]pyrrol-2-one | Rₜ = 1.08/1.09 min (Method A); ES+ 382 (M + H⁺) |
| I-4 | 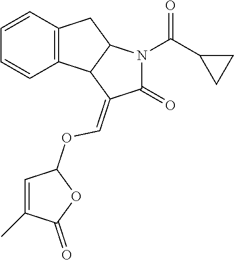 [SMILES: CC1=CC(O/C=C2/C(=O)N(C(=O)C3CC3)C3Cc4ccccc4C23)OC1=O] | (1E)-3- (cyclopropanecarbonyl)-1- [(4-methyl-5-oxo-2H-furan- 2-yl)oxymethylene]-4,8b- dihydro-3aH-indeno[2,1- b]pyrrol-2-one | Rₜ = 1.00/1.01 min (Method A); ES+ 366 (M + H⁺) |
| I-8 | 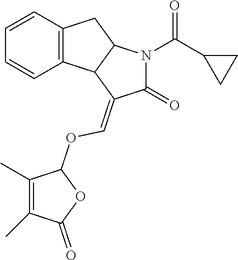 [SMILES: CC1=C(C)C(O/C=C2/C(=O)N(C(=O)C3CC3)C3Cc4ccccc4C23)OC1=O] | (1E)-3- (cyclopropanecarbonyl)-1- [(3,4-dimethyl-5-oxo-2H- furan-2-yl)oxymethylene]- 4,8b-dihydro-3aH- indeno[2,1-b]pyrrol-2-one | Rₜ = 1.04/1.05 min (Method A); ES+ 380 (M + H⁺) |
| I-18 | 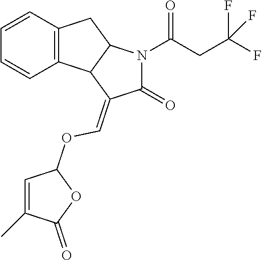 [SMILES: CC1=CC(O/C=C2/C(=O)N(C(=O)CC(F)(F)F)C3Cc4ccccc4C23)OC1=O] | (1E)-1-[(4-methyl-5-oxo-2H- furan-2-yl)oxymethylene]-3- (3,3,3-trifluoropropanoyl)- 4,8b-dihydro-3aH- indeno[2,1-b]pyrrol-2-one | Rₜ = 1.03 min (Method A); ES+ 408 (M + H⁺) |
| I-20 | 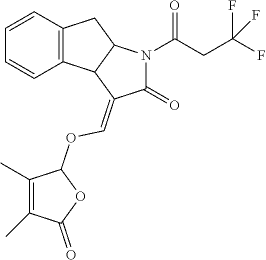 [SMILES: CC1=C(C)C(O/C=C2/C(=O)N(C(=O)CC(F)(F)F)C3Cc4ccccc4C23)OC1=O] | (1E)-1-[(3,4-dimethyl-5-oxo- 2H-furan-2- yl)oxymethylene]-3-(3,3,3- trifluoropropanoyl)-4,8b- dihydro-3aH-indeno[2,1- b]pyrrol-2-one | Rₜ = 1.06/1.07 min (Method A); ES+ 422 (M + H⁺) |
| I-25 | 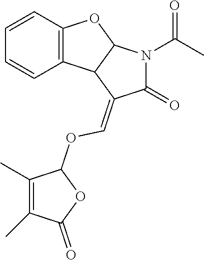 [SMILES: CC(=O)N1C(=O)/C(=C/OC2OC(=O)C(C)=C2C)C2c3ccccc3OC21] | (1E)-3-acetyl-1-[(3,4- dimethyl-5-oxo-2H-furan-2- yl)oxymethylene]-4,8b- dihydro-3aH-indeno[2,1- b]pyrrol-2-one | Rₜ = 0.90/0.91 min (Method A); ES− 244 (M − butenolide/2-hydroxy-3,4- dimethyl-2H-furan-5-one) |
| I-21 | 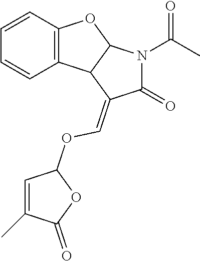 [SMILES: CC(=O)N1C(=O)/C(=C/OC2C=C(C)C(=O)O2)C2c3ccccc3OC21] | (1E)-3-acetyl-1-[(4-methyl- 5-oxo-2H-furan-2- yl)oxymethylene]-4,8b- dihydro-3aH-indeno[2,1- b]pyrrol-2-one | Rₜ = 0.88/0.90 min (Method A); ES+ 342 (M + H⁺) |
| I-89 | 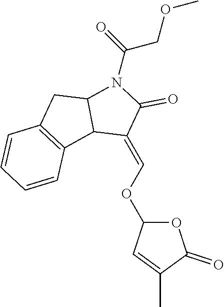 [SMILES: COCC(=O)N1C(=O)/C(=C/OC2C=C(C)C(=O)O2)C2c3ccccc3CC21] | (1E)-3-(2-methoxyacetyl)-1- [(4-methyl-5-oxo-2H-furan- 2-yl)oxymethylene]-4,8b- dihydro-3aH-indeno[2,1- b]pyrrol-2-one | Rₜ = 0.88/0.90 min (Method A); ES+ 370 (M + H⁺) |
| I-90 | 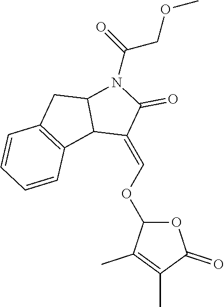 [SMILES: COCC(=O)N1C(=O)/C(=C/OC2OC(=O)C(C)=C2C)C2c3ccccc3CC21] | (1E)-3-(2-methoxyacetyl)-1- [(3,4-dimethyl-5-oxo-2H- furan-2-yl)oxymethylene]- 4,8b-dihydro-3aH- indeno[2,1-b]pyrrol-2-one | Rₜ = 0.92/0.94 min (Method A); ES+ 384 (M + H⁺) |
| I-91 | 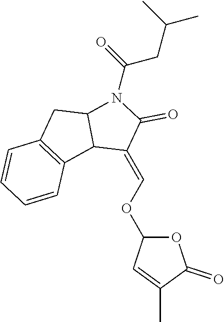 [SMILES: CC1=CC(O/C=C2/C(=O)N(C(=O)CC(C)C)C3Cc4ccccc4C23)OC1=O] | (1E)-1-[(4-methyl-5-oxo-2H- furan-2-yl)oxymethylene]-3- (3-methylbutanoyl)-4,8b- dihydro-3aH-indeno[2,1- b]pyrrol-2-one | Rₜ = 1.08/1.09 min (Method A); ES+ 382 (M + H⁺) |
| I-92 | 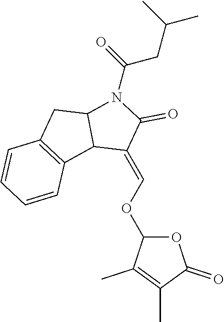 [SMILES: CC1=C(C)C(O/C=C2/C(=O)N(C(=O)CC(C)C)C3Cc4ccccc4C23)OC1=O] | (1E)-1-[(3,4-dimethyl-5-oxo- 2H-furan-2- yl)oxymethylene]-3-(3- methylbutanoyl)-4,8b- dihydro-3aH-indeno[2,1- b]pyrrol-2-one | Rₜ = 1.11/1.13 min (Method A); ES+ 396 (M + H⁺) |
| I-93 | 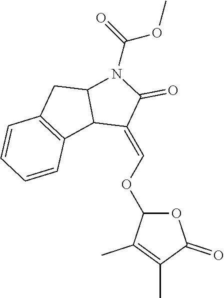 [SMILES: COC(=O)N1C(=O)/C(=C/OC2OC(=O)C(C)=C2C)C2c3ccccc3CC21] | methyl (1E)-1-[(3,4- dimethyl-5-oxo-2H-furan-2- yl)oxymethylene]-2-oxo- 4,8b-dihydro-3aH- indeno[2,1-b]pyrrole-3- carboxylate | Rₜ = 0.92/0.93 min (Method A); ES+ 370 (M + H⁺) |

### BIOLOGICAL EXAMPLES

Comparative biological studies were conducted on compounds according to the invention: Compounds of formula (I) and structurally-related compounds known from the prior art: Compounds (Z) disclosed in WO2012/080115.

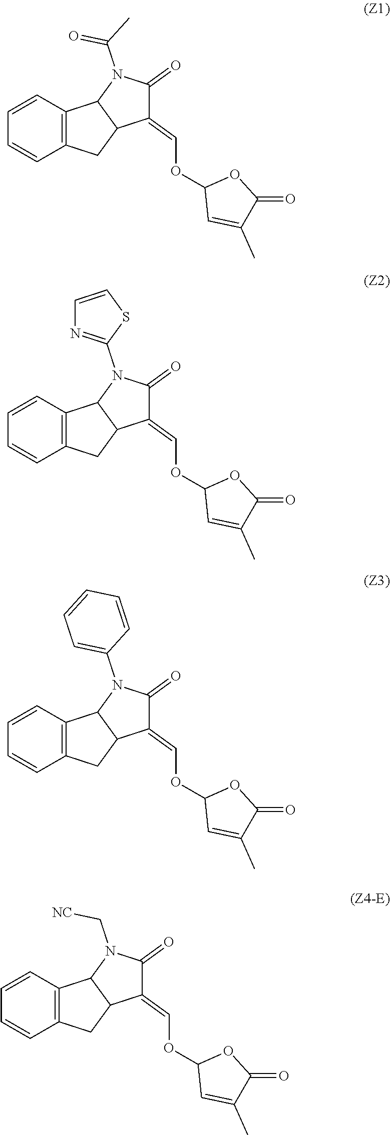 [SMILES: CC(=O)N1C(=O)/C(=C/OC2C=C(C)C(=O)O2)C2Cc3ccccc3C21 ; CC1=CC(O/C=C2/C(=O)N(c3nccs3)C3c4ccccc4CC23)OC1=O ; CC1=CC(O/C=C2/C(=O)N(c3ccccc3)C3c4ccccc4CC23)OC1=O ; CC1=CC(O/C=C2/C(=O)N(CC#N)C3c4ccccc4CC23)OC1=O]

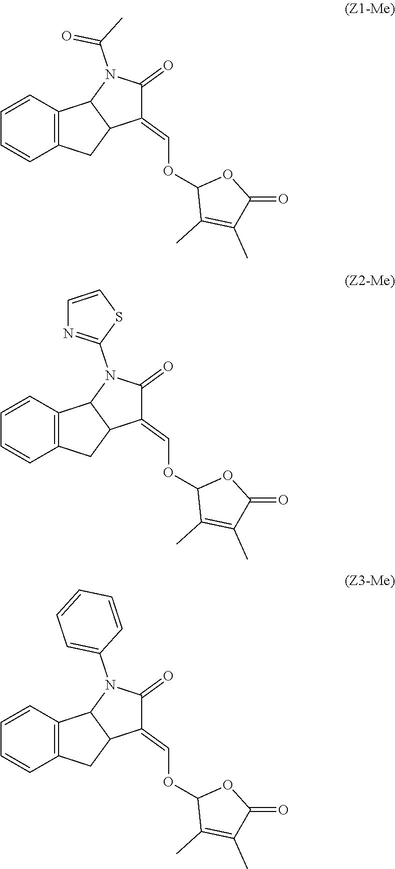 [SMILES: CC(=O)N1C(=O)/C(=C/OC2OC(=O)C(C)=C2C)C2Cc3ccccc3C21 ; CC1=C(C)C(O/C=C2/C(=O)N(c3nccs3)C3c4ccccc4CC23)OC1=O ; CC1=C(C)C(O/C=C2/C(=O)N(c3ccccc3)C3c4ccccc4CC23)OC1=O]

### Example B1: Differential Scanning Fluorometry (DSF)

Strigolactone receptor binding studies were undertaken for the compounds of the present invention. Preparation of the maize strigolactone D14 receptor was conducted by cloning gene ID Zm00001d048146 into the pET SUMO expression vector and transforming into BL21(DE3) One ShotR E. coli cells. The transformed cells were cultured to express the D14 receptor protein, which was then purified via his tag purification.

For the DSF assay, 2 μg of purified D14 receptor protein was used in a reaction volume of 25 μl together with 25x Sypro Orange dye, 5× concentrated phosphate buffer and ddH₂O per well of a 96 well plate. The compounds of the present invention were dissolved in DMSO and tested at a final concentration of 5% DMSO.

Thermal shift is a measure of the difference in temperature (ΔT) required to denature a protein with and without a ligand; this provides an indication of the stabilization or destabilization effect caused by the ligand due to ligand-protein binding. To assess the thermal shift, a CFX Connect Real-Time PCR Detection System (Biorad) was used. After an initial 1 min incubation at 20° C. samples were heat denatured using a linear 20° C.-96° C. gradient, at a rate of 0.5° C./30 sec. Compounds were tested in triplicate at a concentration of 200 μM and a protein/DMSO control was included in every plate to calculate the thermal shift. The results in Table 2 are an average of the 3 replicates.

TABLE 6
Thermal shift (ΔT) of inventive compounds
(I) compared with prior art compounds
(Z) on maize strigolactone receptor D14

| | Rate | ΔT (% |
|---|---|---|
| Compound | (μM) | vs control) |
| I-1 | 50 | 5.6 |
| | 12.5 | 5.5 |
| I-5 | 50 | 4.5 |
| | 12.5 | 2.9 |
| Z1 | 50 | 5.5 |
| | 12.5 | 0.7 |
| Z1-Me | 50 | 1.7 |
| | 12.5 | −1.3 |
| I-9 | 50 | 6.9 |
| | 12.5 | 5.1 |
| I-13 | 50 | 4.3 |
| | 12.5 | 1.9 |
| Z2 | 50 | 0.9 |
| | 12.5 | 0.1 |
| Z2-Me | 50 | 5.3 |
| | 12.5 | 4.0 |
| I-10 | 50 | 3.3 |
| | 12.5 | 0.9 |
| Z3 | 50 | 1.4 |
| | 12.5 | 0.0 |
| I-49 | 50 | 2.5 |
| | 12.5 | −0.3 |
| I-2 | 50 | 8.3 |
| | 12.5 | 6.8 |
| I-6 | 50 | 7.0 |
| | 12.5 | 4.5 |
| I-3 | 50 | 8.3 |
| | 12.5 | 7.1 |
| I-7 | 50 | 12.0 |
| | 12.5 | 10.2 |
| I-25 | 50 | 4.5 |
| | 12.5 | 3.2 |

Compound (I) of the present invention exhibited a higher ΔT compared to prior art compounds (Z). This shows that compounds of the present invention unexpectedly have a superior affinity with the maize strigolactone receptor D14 than close structural analogs.

### Example B2: Dark Induced Senescence of Corn Leaf

It is known that strigolactones regulate (accelerate) leaf senescence, potentially through D14 receptor signaling. Compounds of the present invention (I) were compared to structurally-related compounds (Z) in a corn leaf dark induced senescence assay.

Corn plants of variety Multitop were grown in a greenhouse with relative 75% humidity and at 23-25° C. for 6 weeks. 1.4 cm diameter leaf discs were placed into 24-well plates containing a test compounds in a concentration gradient (100 μM-0.0001 μM) at a final concentration of 0.5% DMSO. Each concentration was tested in 12 replicates. Plates were sealed with seal foil. The foil was pierced to provide gas exchange in each well. The plates were placed into the completely dark climatic chamber. Plates were incubated in the chamber with 75% humidity and at 23° C. for 8 days. On days 0, 5, 6, 7 and 8 photographs were taken of each plate, and image analysis conducted with a macro developed using the ImageJ software. The image analysis was used to determine the concentration at which 50% senescence was achieved (IC50), see Table 7. The lower the value, the higher senescence induction potency.

TABLE 7
IC50 of compounds (I)
and (Z) for dark induced
senescence of corn leaf

| Compound | IC50 (μM) |
|---|---|
| I-1 | 0.064 |
| I-5 | 0.024 |
| Z1 | 0.110 |
| Z1-Me | 0.030 |
| I-9 | 0.126 |
| I-13 | 0.0111 |
| Z2 | inactive |
| Z2-Me | inactive |
| I-10 | 0.107 |
| Z3 | 2.47 |
| I-12-E | 1.55 |
| Z4-E | 3.71 |
| I-25 | 0.99 |

Compounds (I) of the present invention exhibited a lower IC50 value than prior art compounds (Z). This shows that compounds of the present invention unexpectedly lead to a superior leaf senescence promotion activity than close structural analogs. Inducing leaf senescence may improve nutrient (such as nitrogen or sugar) recycling and remobilization in plants at appropriate timing.

1. A compound of formula (I) 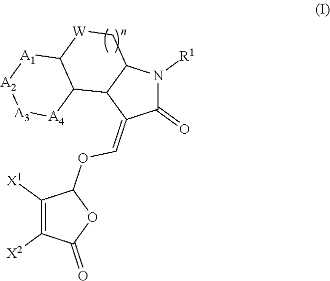
wherein
n is 0, 1 or 2;
W is CH₂ or O;
R¹ is selected from the group consisting of formyl, C₁-C₄ alkylcarbonyl optionally substituted by R², C₃-C₆ cycloalkylcarbonyl optionally substituted by R², C₁-C₄ alkoxycarbonyl optionally substituted by R², C₁-C₄ haloalkylcarbonyl optionally substituted by R², aryl optionally substituted by R², heteroaryl optionally substituted by R², benzyl optionally substituted by R², and acetonitrile,
A₁ to A₄ are each independently selected from the group consisting of a bond, CR², CR²═CR², C(R²)₂, C(R²)₂—C(R²)₂, N, NR′, S and O; wherein A₁ to A₄ together with the atoms to which they are joined form a 4 to 7 membered cycloalkyl, heterocycloalkyl, aryl or heteroaryl; and
wherein each R² is independently selected from the group consisting of hydrogen, halogen, C₁-C₄ alkyl optionally substituted by R⁸, C₂-C₆ alkenyl optionally substituted by R⁸, C₂-C₆ alkynyl optionally substituted by R′, C₁-C₄ alkoxy optionally substituted by R⁸, C₁-C₄ alkoxyalkyl optionally substituted by R′, C₁-C₄ hydroxyalkyl optionally substituted by R′, and C₁-C₄ haloalkyl optionally substituted by R′; or wherein two R² groups are joined to form a 5-6 membered ring;
wherein each R⁸ is independently selected from the group consisting of hydrogen, halogen, C₁-C₄ alkyl, C₂-C₄ alkenyl, C₂-C₄ alkynyl, C₁-C₄ alkoxy, C₃-C₆ cycloalkyl and C₁-C₄ haloalkyl; or two R⁸ groups are joined via —OCH₂O— to forma 5-membered dioxolane ring; and
X¹ and X² are independently selected from the group consisting of hydrogen, C₁-C₄ alkyl, halogen, C₁-C₄ alkoxy, and cyano;
or salts thereof.

2. The compound according to claim 1, wherein A₁ to A₄ together with the atoms to which they are joined form a 4 to 7 membered cycloalkyl, heterocycloalkyl, aryl or heteroaryl ring; and each R² is independently selected from the group consisting of hydrogen, halogen, C₁-C₄ alkyl optionally substituted by R⁸, C₂-C₆ alkenyl optionally substituted by R⁸, C₂-C₆ alkynyl optionally substituted by R⁸, optionally substituted by R⁸, C₁-C₄ alkoxy optionally substituted by R⁸, C₁-C₄ alkoxyalkyl optionally substituted by R⁸, C₁-C₄ hydroxyalkyl optionally substituted by R⁸, and C₁-C₄ haloalkyl optionally substituted by R⁸; or two R² groups are joined to form a 5-6 membered ring.

3. A compound of formula (II) 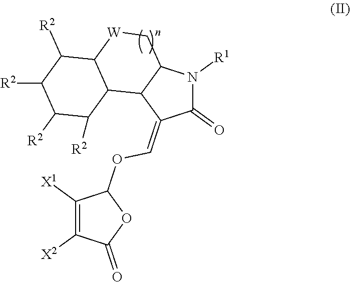
wherein
n is 0, 1 or 2;
W is CH₂ or O;
R¹ is selected from the group consisting of formyl, C₁-C₄ alkylcarbonyl optionally substituted by R², C₃-C₈ cycloalkylcarbonyl optionally substituted by R², C₁-C₄ alkoxycarbonyl optionally substituted by R², C₁-C₄ haloalkylcarbonyl optionally substituted by R², aryl optionally substituted by R², heteroaryl optionally substituted by R² benzyl optionally substituted by R², and acetonitrile;
wherein each R² is independently selected from the group consisting of hydrogen, halogen, C₁-C₄ alkyl optionally substituted by R⁸, C₂-C₆ alkenyl optionally substituted by R⁸, C₂-C₆ alkynyl optionally substituted by R⁸, C₁-C₄ alkoxy optionally substituted by R⁸, C₁-C₄ alkoxyalkyl optionally substituted by R⁸, C₁-C₄ hydroxyalkyl optionally substituted by R′, and C₁-C₄ haloalkyl optionally substituted by R⁸;
or wherein two R² groups are joined to form a 5-6 membered ring;
wherein each R⁸ is independently selected from the group consisting of hydrogen, halogen, C₁-C₄ alkyl, C₂-C₄ alkenyl, C₂-C₄ alkynyl, C₁-C₄ alkoxy, C₃-C₆ cycloalkyl and C₁-C₄ haloalkyl; or two R⁸ groups are joined via —OCH₂O— to forma 5-membered dioxolane ring; and
X¹ and X² are independently selected from the group consisting of hydrogen, C₁-C₄ alkyl, halogen, C₁-C₄ alkoxy, and cyano;
or salts thereof.

4. The compound according to claim 1, wherein ^{X1} and ^{X2} are independently selected from the group consisting of hydrogen, methyl, ethyl and methoxy.

5. The compound according to claim 1, wherein X¹ is hydrogen or methyl and X² is methyl.

6. The compound according to claim 5, wherein X¹ is methyl.

7. The compound according to claim 1, wherein R¹ is selected from the group consisting of formyl, C₁-C₄ alkylcarbonyl, C₃-C₆ cycloalkylcarbonyl, C₁-C₄ alkoxycarbonyl, C₁-C₄ haloalkylcarbonyl, aryl, heteroaryl, benzyl, and acetonitrile.

8. The compound according to claim 7, wherein R¹ is selected from the group consisting of phenyl, C₁-C₄ alkylcarbonyl, heteroaryl, and acetonitrile.

9. A composition comprising a compound according to claim 1, and an agriculturally acceptable formulation adjuvant.

10. A mixture comprising a compound as defined in claim 1, and a further active ingredient.

11. A crop yield enhancing composition, comprising a compound according to claim 1.

12. A method for improving the tolerance of a plant to abiotic stress, promoting seed germination of a plant, or regulating or improving the growth of a plant, wherein the method comprises applying to the plant, plant part, plant propagation material, or plant growing locus a compound according to claim 1.

13. The compound according to claim 3, wherein ^{X1} and ^{X2} are independently selected from the group consisting of hydrogen, methyl, ethyl and methoxy.

14. The compound according to claim 3, wherein X¹ is hydrogen or methyl and X² is methyl.

15. The compound according to claim 14, wherein X¹ is methyl.

16. The compound according to claim 3, wherein R¹ is selected from the group consisting of formyl, C₁-C₄ alkylcarbonyl, C₃-C₆ cycloalkylcarbonyl, C₁-C₄ alkoxycarbonyl, C₁-C₄ haloalkylcarbonyl, aryl, heteroaryl, benzyl, and acetonitrile.

17. The compound according to claim 16, wherein R¹ is selected from the group consisting of phenyl, C₁-C₄ alkylcarbonyl, heteroaryl, and acetonitrile.

18. A composition comprising a compound according to claim 3, and an agriculturally acceptable formulation adjuvant.

19. A mixture comprising a compound as defined in claim 3, and a further active ingredient.

20. A method for improving the tolerance of a plant to abiotic stress, promoting seed germination of a plant, or regulating or improving the growth of a plant, wherein the method comprises applying to the plant, plant part, plant propagation material, or plant growing locus a compound according to claim 3.
